(19) United States
(12) Patent Application Publication
(10) Pub. No.: US 2019/0101609 A1
(43) Pub. Date: Apr. 4, 2019

(54) PREDICTING SOLIDS CONTENT, BOILING POINT INFORMATION, AND MICRO CARBON RESIDUE CONTENT OF EXTRA HEAVY OIL SAMPLES USING LOW-FIELD TIME-DOMAIN NMR

(71) Applicants: SYNCRUDE CANADA LTD. in trust for the owners of, Fort McMurray (CA); the Syncrude Project as such owners exist now and in the future, Fort McMurray (CA)

(72) Inventors: RICHARD PAPROSKI, Edmonton (CA); NATHANIEL DIRK FLIM, Fort Saskatchewan (CA)

(21) Appl. No.: 15/725,120

(22) Filed: Oct. 4, 2017

(51) Int. Cl. G01R 33/58; G01N 24/08; G01N 33/28

(52) U.S. Cl. CPC G01R 33/58; G01N 33/28; G01N 24/081

(57) ABSTRACT

A method for predicting a solids content, boiling point information, and micro carbon residue content of an extra heavy oil test sample involves subjecting extra heavy oil calibration samples to T₁-weighted T₂ low-field NMR pulse sequences, and recording T₁ and T₂ relaxation information to generate a NMR calibration data set. A chemometric partial least squares calibration model is determined to relate the NMR calibration data set to reference values of properties of the calibration samples. Similarly, the extra heavy oil test sample is subjected to the NMR T₁-weighted T₂ low-field NMR pulse sequences, the resulting relaxation information is recorded to generate NMR test data. The properties of the test sample are predicted based on the PLS calibration model and the NMR test data. The test sample may be derived from a process stream for upgrading bitumen, and may contain significant amounts of solids and high boiling point hydrocarbons (e.g., >524° C.).

### FIELD OF THE INVENTION

The present invention relates to use of low-field time domain nuclear magnetic resonance to predict the solids content, boiling point information, and micro carbon residue content of extra heavy oil samples, such as those that may be produced during the upgrading of bitumen recovered from oil sands ore, to relatively lighter, higher quality crude oil.

### BACKGROUND OF THE INVENTION

### Extra Heavy Oil

Extra heavy oil may be produced as intermediate products during the upgrading of bitumen recovered from oil sands ore to relatively lighter, higher quality crude oil. Such extra heavy oil may be characterized by a significant solids content, micro carbon residue content, and/or relatively large amount of hydrocarbon molecules having high boiling point temperatures greater than 524° C. (975° F.). In a vacuum distillation process, boiling point temperatures at or near 524° C. is conventionally used to demarcate between gas oils (boiling point temperature <524° C.) and undistilled hydrocarbon residues (boiling point temperature >524° C.).

### Coker Scrubber Slurry

An example of extra heavy oil is coker scrubber slurry. During the upgrading of bitumen, the non-distillable residue of a vacuum distillation process is spray fed into a reactor section of a fluid coker where it mixes with hot fluidized fine coke particles, and is thereby cracked to form vapors. The vapors then enter a scrubber section of the fluid coker where they are scrubbed to remove coke particles and cooled to condense high boiling liquids. The coker scrubber slurry is the resulting scrubber section bottoms comprising high molecular weight, long chain hydrocarbon molecules in relatively liquid form mixed with fine fluid coke particles typically ranging from 2 to 5 percent by weight, with possible excursions up to 30 percent by weight. (For clarity, the term “fluid” in the expression “fluid coke particles” refers to the formation of such particles in a fluid coker, rather than to the particles being in a fluidized condition.) Part of the coker scrubber slurry may be recycled back to the reactor section of the fluid coker for further processing, while another part of the coker scrubber slurry may be cooled and recycled back to the scrubber section.

It may be also important to know the solids content, boiling point information, and micro carbon residue content of the coker scrubber slurry. High solids content (e.g., in excess of 10 percent by weight) can result in fouling of process lines and erosion of pumps associated with the fluid coker. The <524° C. hydrocarbon fraction is preferably minimized to reduce the recycling of lighter boiling liquids to the reactor section, and thus incrementally improve the yield and/or throughput of the fluid coker. Micro carbon residue content is a measure of the coke forming propensity of the coker scrubber slurry.

Analytical methods exist for determining solids content, boiling point information, and micro carbon residue content. Solids content can be determined by centrifugation-based water and sediment test methods, such as in accordance with ASTM D4007. Another approach is vacuum filtration to measure the weight of solids retained on a filter of a given pore size following dilution of the sample with an appropriate solvent such as toluene.

Boiling point information can be determined by high temperature gas chromatography, such as in accordance with ASTM D7169. However, fine solid particles in the coker scrubber slurry may foul chromatographic equipment. Further, solid particles contribute to the total sample weight, but do not produce a signal in the gas chromatograph giving rise to an apparent missing mass. This apparent missing mass may be erroneously attributed to the unrecovered high boiling point fraction (e.g., >720° C.) of the sample. Removal of such solids by hot filtration prior to analysis may improve the accuracy of this method, but hours may be needed to produce a sufficient amount of filtered sample for analysis.

Micro carbon residue content can be determined by carbon residue micro method after evaporation and pyrolysis of hydrocarbons, such as in accordance with ASTM D4530. However, the coker scrubber slurry contains fine solids that would be included in the total micro carbon residue weight. This would result in over-reporting of the coke-forming propensity of the liquid portion of the coker scrubber slurry. Again, selective removal of such solids by hot filtration prior to analysis may improve the accuracy of this approach, but would be time consuming.

The above analytical methods typically require significant amounts of time and resources. They are not amenable to rapid monitoring of coker scrubber slurry properties, as would be desirable for process control of the fluid coker in real-time, especially during start-up operations or troubleshooting.

### Low-Field Nuclear Magnetic Resonance (Low-Field NMR)

Low-field time domain nuclear magnetic resonance (low-field NMR) may be used to analyze the properties of samples containing hydrogen-bearing molecules. Low-field NMR involves applying an external magnetic field to a sample to align the nuclear spin of hydrogen atoms in the sample, and then applying one or multiple radio frequency pulse(s) to the sample to change the net spin orientation of the hydrogen nuclei relative to the external magnetic field. The nuclei undergo both transverse (T₂) and longitudinal (T₁) relaxation processes as they return to their equilibrium position (i.e., towards alignment with the external magnetic field). As known in the art, relaxation rates depend on several factors, including mobility of the hydrogen nuclei in the sample. Different low field NMR measurements involving various radio frequency pulse sequences can be performed to study relaxation behavior to gain insight into the properties and/or composition of samples. Low-field NMR measurements can be simple to perform, quick, non-destructive, and can require no solvents. They can also be more accurate and precise, and less susceptible to technician bias than some other analytical methods.

### Low-Field NMR for Analysis of Hydrocarbon Mixtures

U.S. Patent Application Publication No. 2003/0128032 (Heaton et al.) discloses a method of determining a molecular property (e.g., molecular size, carbon number, or weight) of each constituent in a mixture of hydrocarbons (e.g., crude oils in a geological formation) based on NMR data (e.g., relaxation times, or diffusion rates) acquired with a NMR logging device. The determination of the molecular property involves correlating the NMR data with an effective viscosity of the constituent. Heaton et al. caution that with the numerous assumptions involved in their approach, “any resulting ‘carbon number’ (or molecular size) distribution should probably be regarded as an approximate indicator rather than a definitive and accurate breakdown of molecular composition”. Given that boiling point temperatures depend on other factors beyond molecular weight or carbon number (i.e., elemental composition and structure), it is unlikely that boiling range information could be accurately determined from data produced by this approach.

U.S. Patent Application Publication No. 2010/0271019 (Anand et al.) discloses a NMR tool with a pressure cell on or near the earth's surface, in which live crude oil is analyzed. A non-linear mapping function (e.g., Gaussian radial basis function) is created using a database of live crude oil properties and existing NMR measurements. A fluid property (e.g. viscosity, molecular composition, and SARA fractions) is predicted based on NMR data acquired using the NMR tool and the mapping function. However, this method is specific to live crude oil samples, and would not permit for fast analysis times since the live oil sample is equilibrated for one to five days in the pressure cell before NMR measurements are performed. It is unclear how accurate boiling point information could be determined from the data produced by this approach.

U.S. Patent Application Publication No. 2013/0103627 (Maddinelli et al.) discloses a method for using T₂ NMR relaxation curves and neural networks to predict various crude oil sample properties (e.g., carbonaceous residue content), or alternatively, distillation information of the crude oil. Specifically, the T₂ NMR relaxation curves are first plotted on a logarithmic scale, and then intersected with grid lines to produce selected values with a constant time distance between 0.1 and 1 milliseconds. These selected NMR values and known training sample properties are processed using a neural network and optimized with genetic algorithms. Of note, excessively short distance between the lines of the grid creates an input overload for the neural network with the consequent risk of overlearning, thus jeopardizing the accuracy of the predicted value. Examples of the method are applied to crude oil samples having API gravity values of 19 degrees or greater. (API gravity value is an inverse measure of liquid density relative to water, with a value greater than 10 degrees indicating a density less than that of water, and a value of less than 10 degrees indicating a density greater than water.) This approach does not address samples containing significant solids content (e.g. >1% by weight). As is known in the art, a significant concentration of solids in extra heavy oils can alter the NMR relaxation information obtained through restricted diffusion and/or surface relaxation processes.

PCT International patent application publication WO 2016/023984 A1 (Adam-Berret et al.) discloses a method for determining a process monitoring parameter that can be used to predict the stability of asphaltenes (e.g., risk of asphaltene precipitation) of a petroleum product containing asphaltenes (e.g., vacuum residues from the distillation of crude oil containing 2 to 25% by weight of asphaltenes) using low field NMR based on a ratio of mean T₁ relaxation time to mean T₂ relaxation time. Adam-Berret et al. recognize the importance of maintaining this ratio below a target operating value in order to avoid asphaltene precipitation. Adam-Berret et al. recognize a relationship between this ratio and a percent “conversion” of the asphaltenes within the >525° C. cut of both the feed and the effluent [((Wt % asphaltenes in the >525° C. cut of the feed)−(Wt % asphaltenes in the >525° C. cut of the effluent))/(Wt % asphaltenes in the >525° C. cut of the feed)].

PCT International patent application publication WO 2017/030559 A1 (Sandor et al.) discloses using a downhole NMR tool to indirectly measure asphaltene concentration in crude oils having API gravity values of about 20 to 41 degrees. More specifically, a measured NMR property of an unknown sample is applied to a mathematical regression for asphaltene concentration in crude oil as a function of an NMR property, in accordance with an equation relating asphaltene concentration to intrinsic viscosity of the crude oil sample.

The following publications disclose using NMR to analyze the viscosity of bitumen mixtures: Afsahi, B., Kantzas, A. “Advances in diffusivity measurement of solvents in oil sands” (2007) Journal of Canadian Petroleum Technology, 46 (11) 56-61; Wen, Y., Kantzas, A., “Evaluation of heavy oil/bitumen-solvent mixture viscosity models” (2006) Journal of Canadian Petroleum Technology, 45 (4) 56-61; Wen, Y. W., Kantzas, A., “Monitoring bitumen-solvent interactions with low-field nuclear magnetic resonance and X-ray computer-assisted tomography” (2005) Energy and Fuels, 19 (4) 1319-1326; Wen, Y., Bryan, J., Kantzas, A, “Estimation of diffusion coefficients in bitumen solvent mixtures as derived from low field NMR spectra” (2005) Journal of Canadian Petroleum Technology, 44 (4) 29-34; Wen, Y., Bryan, J., Kantzas, A., “Evaluation of bitumen-solvent properties using low field NMR” (2005) Journal of Canadian Petroleum Technology, 44 (4) 22-28; Bryan, J., Moon, D., Kantzas, A., “In situ viscosity of oil sands using low field NMR” (2005) Journal of Canadian Petroleum Technology, 44 (9) 23-29. Yang, Z., Hirasaki, G. J. “NMR measurement of bitumen at different temperatures” (2008) Journal of Magnetic Resonance, 192 (2) 280-293; and Bryan, J., Kantzas, A., Bellehumeur, C. “Oil-viscosity predictions from low-field NMR measurements” (2005) SPE Reservoir Evaluation and Engineering, 8 (1) 44-52; and Bryan, J., Mirotchnik, K., Kantzas, A. “Viscosity determination of heavy oil and bitumen using NMR relaxometry” (2003) Journal of Canadian Petroleum Technology, 42 (7) 29-34.

### SUMMARY OF THE INVENTION

There remains a need in the art for a method of rapidly quantifying the solids content, boiling point information, and micro carbon residue content of extra heavy oil samples, such as those derived from processing streams during the upgrading of bitumen recovered from oil sands ore to lighter, higher quality crude oils.

The present invention uses a low-field time-domain nuclear magnetic resonance (NMR) instrument to predict the solids content, boiling point information, and micro carbon residue content of extra heavy oil samples, with reasonably good accuracy. The NMR instrument is used to simultaneously collect T₁ and T₂ relaxation information from calibration samples where such properties are known or determinable. Partial least squares chemometric calibration models that relate the relaxation information to such properties of the calibration samples are then determined. The NMR instrument is then used to simultaneously collect T₁ and T₂ relaxation information from a test sample. The calibration model is applied to the relaxation information of the test sample to predict such properties of the test sample. The present invention may allow for rapid predictions of such properties for extra heavy oil samples having API Gravity values of less than or equal to 10 degrees, solids content greater than 1, 5, 10, or 15 percent, and micro carbon residue content greater than 1, 5, 10, 20, 30 or 40 percent. Examples of such extra heavy oil samples include fluid coker scrubber slurry, or residues of an atmospheric distillation column, a vacuum distillation column, or an ebullated bed hydrocracking reactor.

In one aspect, the present invention may comprises a method for predicting at least one property of an extra heavy oil test sample using an NMR instrument, wherein the at least one property comprises one or a combination of a solids content, boiling point information, and a micro carbon residue (MCR) content. The method comprises the steps of:
(a) providing a plurality of extra heavy oil calibration samples, wherein each of the calibration samples has a reference value associated with each of the at least one property;
(b) subjecting each of the calibration samples to a T₁-weighted T₂ low-field NMR pulse sequence, and simultaneously recording T₁ and T₂ relaxation information to generate a NMR calibration data set;
(c) for each of the at least one property, determining a chemometric partial least squares (PLS) calibration model relating the NMR calibration data set to the reference values of the property of the calibration samples;
(d) subjecting the test sample to the T₁-weighted T₂ low-field NMR pulse sequence, and simultaneously recording T₁ and T₂ relaxation information to generate NMR test data; and
(e) predicting the at least one property of the test sample based on the PLS calibration model and the NMR test data.

In an embodiment of the method, the extra heavy oil test sample is obtained from a process stream for upgrading bitumen recovered from oil sands ore to lighter crude oil. As non-limiting examples, the extra heavy oil test sample may comprise one or a combination of a fluid coker scrubber slurry, a residue of an atmospheric distillation column, a residue of a vacuum distillation column, or a residue of an ebullated bed hydrocracking reactor.

In an embodiment of the method, the method further comprises the steps of selecting the associated reference values for the at least one property, and preparing the calibration samples with the selected associated reference values.

In an embodiment of the method, the at least one property comprises the solids content. In such embodiment, determining the PLS calibration model may comprise relating reference values for non-solid content in the calibration samples to the NMR T₁-weighted T₂ signal data indicative of non-solids content in the calibration samples. In such embodiment, predicting the solids content of the test sample may comprise subtracting the non-solids content of the test sample as predicted by the PLS calibration model from a weight of a whole of the test sample. In such embodiment, the method may further comprise the step of determining the reference values for the solids content of the calibration samples by subjecting the calibration samples or subsamples thereof to extraneous matter vacuum filtration following dilution with an appropriate solvent to reduce the viscosity of the calibration samples or subsamples thereof.

In an embodiment of the method, the at least one property comprises boiling point information. In such embodiment, the boiling point information may comprise a concentration, by weight, of hydrocarbon content having a boiling point temperature less than or greater than at least one specified temperature, such as 524° C. In such embodiment, the method may further comprise the step of determining the reference values for the boiling point information of the calibration samples by subjecting the calibration samples or subsamples thereof to simulated distillation analysis by gas chromatography. Optionally, such gas chromatography may follow removal of solids from the calibration samples or subsamples thereof by hot filtration.

In an embodiment of the method, the at least one property comprises the MCR content. In such embodiment, determining the PLS calibration model may comprise relating reference values for non-MCR content in the calibration samples to the NMR T₁-weighted T₂ signal data indicative of non-MCR content in the calibration samples. In such embodiment, predicting the MCR content of the test sample may comprise subtracting the non-MCR content of the test sample as predicted by the PLS calibration model from a weight of a whole of the test sample. The MCR content of a liquid portion of the test sample can be calculated by subtracting a solids content of the test sample from the predicted MCR content of the test sample as a whole. In such embodiment, the method may further comprise the step of determining the reference values for the MCR content of the calibration samples by subjecting the calibration samples or subsamples thereof to micro carbon residue analysis by measuring the residual weight of the calibration samples or subsamples thereof after evaporation and pyrolysis of hydrocarbon material at 500° C. under a nitrogen atmosphere. Optionally, the evaporation and pyrolysis may follow hot filtration of the calibration samples or subsamples thereof to produce a solids-free, liquid sub-sample amenable for MCR analysis without the contribution of solids to the MCR result.

In an embodiment of the method, the at least one property comprises the combination of solids content, boiling point information, and/or MCR content.

In an embodiment of the method:
(a) the method further comprises removing at least a fraction of solids from the calibration samples to produce liquid portions of the calibration samples;
(b) step (b) of the method described above comprises subjecting the liquid portions of the calibration samples to the T₁-weighted T₂ low-field NMR pulse sequence;
(c) the method further comprises removing at least a fraction of solids from the test sample to produce a liquid portion of the test sample;
(d) step (d) of the method described above comprises subjecting the liquid portion of the test sample to the T₁-weighted T₂ low-field NMR pulse sequence; and
(e) step (e) of the method described above comprises predicting the at least one property of the test sample for the liquid portion of the test sample.

In such embodiment, the at least one property may comprise the boiling point information or the MCR content. In such embodiment, the liquid portions of the calibration samples may comprise liquid filtrates produced by hot filtration of the calibration samples, and the liquid portion of the test sample may comprise the liquid filtrate produced by hot filtration of the test sample.

In an embodiment of the method, when the calibration samples and the test sample are subjected to the T₁-weighted T₂ low-field NMR pulse sequence, the calibration samples and the test samples are contained in glass containers. In such embodiment, the glass container comprises a lower part holding the calibration sample or the test sample, and an upper part placed over a top of the lower part to prevent heat loss from a top portion of the calibration sample or the test sample. In such embodiment, the upper and lower parts may be glass vials.

In an embodiment of the method, the calibration samples, the test sample, and an NMR probe of the NMR instrument are heated to a temperature of at least 40° C., when the calibration samples and the test sample are subjected to the T₁-weighted T₂ low-field NMR pulse sequence. In such embodiment, the temperature may be at least 70° C.

In an embodiment of the method, the T₁-weighted T₂ low-field NMR pulse sequence is such that there are 50 transverse relaxation echoes spaced 0.04 ms apart, acquired at 28 T₁ points, exponentially spread from 5 ms through 1000 ms, with 16 scans averaged together to improve the signal to noise ratio, resulting in a measurement time of less than 2 minutes.

### BRIEF DESCRIPTION OF THE DRAWINGS

FIG. 1A shows raw T₁-weighted T₂ low-field NMR signals, plotted by data point, for samples of various upgrading process streams, in accordance with Example 1;

FIG. 1B shows a sub-set of the T₁-weighted T₂ low-field NMR signals of FIG. 1A plotted by data point, for clarity;

FIG. 2 shows the FID-relaxation NMR signals for samples of mixtures comprising Unitar bitumen, >524° C. hydrocarbon material, and solid fluid coke particles, in accordance with Example 2;

FIG. 3 shows Table 1, which summarizes the composition of simulated coker slurry samples, in accordance with Example 3;

FIG. 4A shows the raw T₁-weighted T₂ low-field NMR signals, plotted by data point, for some of the samples of FIG. 3, Table 1;

FIG. 4B shows a sub-set of the T₁-weighted T₂ low-field NMR signals of FIG. 4A plotted by data point, for clarity;

FIG. 5 compares the >524° C. hydrocarbon content predicted by a PLS calibration model based on NMR signals, to reference values, for the samples of FIG. 3, Table 1;

FIG. 6 compares the <524° C. hydrocarbon content predicted by a PLS calibration model based on NMR signals, to reference values, for the samples of FIG. 3, Table 1;

FIG. 7 compares the amount of solid fluid coke particles predicted by a PLS calibration model based on NMR signals, to reference values, for the samples of FIG. 3, Table 1;

FIG. 8 shows Table 2, which summarizes statistical measures of the difference between the predicted and reference values for the properties compared in FIGS. 5 to 7;

FIG. 9A shows a raw T₁-weighted T₂ low-field NMR signal, plotted by time, for a fluid coker scrubber slurry sample at 40° C., in accordance with Example 4;

FIG. 9B shows the T₁-weighted T₂ low-field NMR signal of FIG. 9A, plotted by data point;

FIGS. 10A, 10B, 10C, and 10D show the PLS calibration model loadings for determining the non-solids content, the <524° C. hydrocarbon content, the >524° C. hydrocarbon content, and the non-MCR content, respectively, from NMR signals such as that of FIG. 9B;

FIG. 11 shows Table 3, which summarizes statistical measures of the difference between values predicted by PLS calibration models, and values determined by other analytical methods, for properties of fluid coker scrubber slurry samples, in accordance with Example 4;

FIGS. 12A and 12B compare the solids content for fluid coker scrubber slurry test set samples as predicted by a non-solids PLS calibration model based on calibration samples at 40° C. and 70° C., respectively, to reference values determined by vacuum filtration analysis, in accordance with Example 4;

FIGS. 13A to 13D compare the <524° C. hydrocarbon content, on a “liquid only” basis (FIGS. 13A and 13C) and “whole sample” basis (FIGS. 13B and 13D) for fluid coker scrubber slurry test samples, as predicted by PLS calibration models based on calibration samples at 40° C. (FIGS. 13A and 13B) and 70° C. (FIGS. 13C and 13D), to reference values determined by high temperature simulated distillation of a filtrate, in accordance with Example 4;

FIGS. 14A to 14D compare the >524° C. hydrocarbon content, on a “liquid only” basis (FIGS. 14A and 14C) and “whole sample” basis (FIGS. 14B and 14D) of fluid coker scrubber slurry test samples, as predicted by PLS calibration models based on calibration samples at 40° C. (FIGS. 14A and 14B) and 70° C. (FIGS. 14C and 14D), to reference values determined by high temperature simulated distillation on a filtrate, in accordance with Example 4;

FIGS. 15A to 15B compare the MCR content, on a “liquid only” and “whole sample” basis, respectively, of fluid coker scrubber slurry test samples, as predicted by a non-MCR PLS calibration model based on calibration samples at 40° C., to reference values determined by micro carbon residue analysis, in accordance with Example 4;

FIGS. 16A to 16B compare the MCR content, on a “liquid only” and “whole sample” basis, respectively, of fluid coker scrubber slurry test samples, as predicted by a MCR PLS calibration model based on calibration samples at 40° C., to reference values determined by micro carbon residue analysis, in accordance with Example 4;

FIGS. 17A to 17B compare the MCR content, on a “liquid only” and “whole sample” basis, respectively, of fluid coker scrubber slurry test samples, as predicted by a non-MCR PLS calibration model based on calibration samples at 70° C., to reference values determined by micro carbon residue analysis, in accordance with Example 4;

FIG. 18 compares the MCR content, on a “liquid only” basis of fluid coker scrubber slurry test samples, as predicted by a MCR PLS calibration model based on calibration samples at 70° C., to reference values determined by micro carbon residue analysis, in accordance with Example 4;

FIG. 19 shows Table 4, which summarizes statistical measures for 10 NMR analyses of 3 fluid coker slurry test samples at 40° C., for <524° C. hydrocarbon content, >524° C. hydrocarbon content, and MCR content as predicted by a non-MCR PLS calibration model, in accordance with Example 4;

FIG. 20 shows Table 5, which summarizes statistical measures of analyses for 10 sub-samples of a single fluid coker slurry test sample at 40° C., for <524° C. hydrocarbon content, >524° C. hydrocarbon content, and MCR content as predicted by a non-MCR PLS calibration model, in accordance with Example 4;

FIG. 21A shows a raw T₁-weighted T₂ low-field NMR signal, plotted by time, for a fluid coker scrubber slurry test sample at 40° C., in accordance with Example 5;

FIG. 21B shows the T₁-weighted T₂ low-field NMR signals of FIG. 21A, plotted by data point;

FIGS. 22A, 22B, 22C, and 22D show the PLS calibration model loading for determining the non-solids content, the <524° C. hydrocarbon material content, the >524° C. hydrocarbon material, and the non-MCR content, respectively, from NMR signals such as that of FIG. 21B;

FIG. 23 shows Table 6, which summarizes statistical measures of the difference between values predicted by PLS calibration models, and values determined by other analytical methods, for various properties of fluid coker scrubber slurry test samples, in accordance with Example 5;

FIGS. 24A and 24B compare the solids content for fluid coker scrubber slurry test samples as predicted by a non-solids PLS calibration model based on calibration samples at 40° C. and 70° C., respectively, to reference values determined by vacuum filtration analysis, in accordance with Example 5;

FIGS. 25A to 25D compare the <524° C. hydrocarbon content, on a “liquid only” basis (FIGS. 25A and 25C) and “whole sample” basis (FIGS. 25B and 25D) for fluid coker scrubber slurry test samples, as predicted by PLS calibration models based on calibration samples at 40° C. (FIGS. 25A and 25B) and 70° C. (FIGS. 25C and 25D), to reference values determined by high temperature simulated distillation of a filtrate, in accordance with Example 5;

FIGS. 26A to 26D compare the >524° C. hydrocarbon content, on a “liquid only” basis (FIGS. 26A and 26C) and “whole sample” basis (FIGS. 26B and 26D) of fluid coker scrubber slurry test samples, as predicted by PLS calibration models based on calibration samples at 40° C. (FIGS. 26A and 26B) and 70° C. (FIGS. 26C and 26D), to reference values determined by high temperature simulated distillation on a filtrate, in accordance with Example 5;

FIGS. 27A to 27B compare the MCR content, on a “liquid only” and “whole sample” basis, respectively, of fluid coker scrubber slurry test samples, as predicted by a non-MCR PLS calibration model based on calibration samples at 40° C., to reference values determined by micro carbon residue analysis, in accordance with Example 5;

FIGS. 28A to 28B compare the MCR content, on a “liquid only” and “whole sample” basis, respectively, of fluid coker scrubber slurry test samples, as predicted by a non-MCR PLS calibration model based on calibration samples at 70° C., to reference values determined by micro carbon residue analysis, in accordance with Example 5;

FIG. 29 shows Table 7, which summarizes statistical measures for 10 analyses of 3 fluid coker slurry test samples at 40° C., for <524° C. hydrocarbon content, >524° C. hydrocarbon content, and MCR content as predicted by a PLS calibration model based, in accordance with Example 5;

FIG. 30 shows a flow chart of an embodiment of a method of the present invention.

### DETAILED DESCRIPTION OF THE PREFERRED EMBODIMENTS

### Definitions

As used herein, the following expressions have the following meanings.

“Extra heavy oil” refers to a composition comprising hydrocarbons with boiling point temperatures above 524° C., and having an API gravity value of less than or equal to 10 degrees, as determined in accordance with the following formula, where SG is the specific gravity of the heavy oil test sample at a temperature of 60° F. (about 15.6° C.):

$$API Gravity = \frac{141.5}{SG} - 131.5$$

In embodiments, extra heavy oil may comprise solids. In embodiments, extra heavy oil may have a solids content greater than greater than 1, 5, 10, or 15 percent. In embodiments, the solids in the extra heavy oil may include fine petroleum coke-based particles such as those produced in a fluid coker, and/or mineral particles. In embodiments, extra heavy oil may have a micro carbon residue content greater than 1, 5, 10, 20, 30 or 40 percent. In embodiments, the extra heavy oil may be sourced from a bitumen upgrading process stream including, without limitation, a residue of an atmospheric distillation column, a residue of a vacuum distillation column, a fluid coker scrubber slurry, or a residue of hydrocracking reactor (e.g., an ebullated bed LC-Finer hydrocracker).

“Solids content” refers to the concentration, by weight, of solid material in the extra heavy oil sample. As an example, solids content may be measured by water and sediment test methods by centrifugation, such as in accordance with ASTM D4007, or by vacuum filtration after dilution with an appropriate solvent (e.g., toluene) to measure the weight of solids retained on a filter of a given pore size.

“Boiling point information” refers to the concentration, by weight, of the non-solid hydrocarbon content having a boiling point temperature less than and/or greater than a specified temperature in the extra heavy oil sample. As an example, boiling point information may be measured by various distillation methods or simulated distillation by high temperature gas chromatography in accordance with ASTM D7169. In some embodiments, the specified temperature may be 524° C. (975° F.). “524° C.+”, “>524° C.”, and similar expressions refer to compositions having a boiling point temperature greater than or equal to 524° C., whereas “524° C.−”, “<524° C.” and similar expressions refer to compositions having a boiling point temperature less than 524° C.

“Micro carbon residue content” or “MCR content” refers to a quantifiable indicator of the coke-forming propensity of the extra heavy oil sample. As an example, MCR content may be measured by the carbon residue micro method by weighing the carbonaceous residue following evaporation and pyrolysis of hydrocarbons, in accordance with ASTM D4530.

“Whole sample basis” refers to the concentration, by weight, of a component in the extra heavy oil sample, as determined by reference to the weight of the entire extra heavy oil sample, including its solids content.

“Liquid only basis” refers to the concentration, by weight, of a component in the extra heavy oil sample, as determined by reference to the weight of the extra heavy oil sample, excluding its solids content. As an example, the solids content that is excluded from determining the “liquid only basis” concentration of a component may include material that does not pass through a filter having a pore size of about 1.6 microns.

“Unitar bitumen” refers to an Athabasca-mined bitumen material previously prepared and sub-sampled for an inter-laboratory study, with an initial boiling point of about 244° C. and containing about 59.5 percent by weight of >524° C. hydrocarbon material according to high temperature simulated distillation analysis.

### Overview

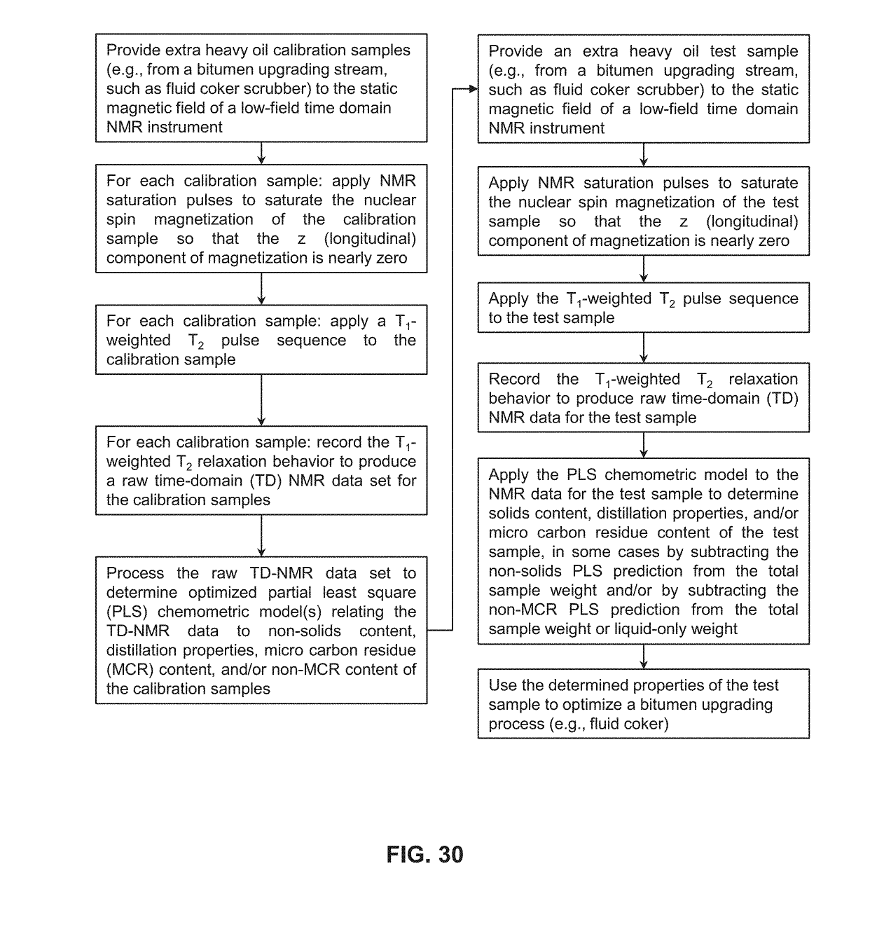

The present invention uses low-field time-domain nuclear magnetic resonance (low-field TD-NMR) instruments to analyze an extra heavy oil test sample to predict one or a combination of the following properties of the sample: solids content, boiling point information of the non-solid hydrocarbon content; and micro carbon residue content. FIG. 30 shows a flow chart of an embodiment of a method of the present invention.

Embodiments of the invention are illustrated by way of examples described below. In the examples, the NMR instrument used to perform low-field TD-NMR measurements was a Bruker Minispec mq10™ NMR instrument (Bruker BioSpin Ltd., Milton, Ontario, Canada). More particularly, the NMR instrument was used to subject extra heavy oil samples to T₁-weighted T₂ measurement NMR pulse sequences to collect raw TD-NMR data sets including both T₁ and T₂ relaxation information.

The TD-NMR measurement process involves using the NMR instrument to subject the sample to an externally applied magnetic field to align the nuclear spin of hydrogen atoms in the sample. A series of 90° radio-frequency pulses are initially applied to the sample to saturate the magnetization in the +Z axis. The sample is then subjected to a T₁-weighted T₂ sequence of radio-frequency pulses. Transverse relaxation (T₂) echo trains are recorded after incremental longitudinal (T₁) relaxation to produce a raw TD-NMR data set for the sample. Both the longitudinal (T₁) and transverse (T₂) relaxation behavior can be observed and recorded simultaneously.

In the examples that follow, the noted operating parameters of the NMR instrument have the following meanings:
“probe dead-time” refers to the duration, in milliseconds, during which the NMR probe is incapable of receiving data, beginning at the end of a NMR pulse;
“scans” refers to the number of replicate T₁-weighted T₂ measurements that are averaged together to generate a raw NMR signal for a given sample; and
“NMR analysis time” refers to the total time required by the NMR instrument to subject a sample to the replicate T₁-weighted T₂ NMR pulse sequence scans.

The NMR instrument should be maintained in a clean and dry, temperature controlled environment. The instrument is sensitive to swings in ambient temperature and should not be positioned in the direct path an air duct that periodically delivers either warm or cool air. In general, better accuracy and precision can be expected when both ambient and test sample temperatures are tightly controlled.

In Examples 3 to 5 that follow, the partial least squares (PLS) chemometric models were determined using OPUS™ software version 7.0129 (Bruker BioSpin Ltd., Milton, Ontario, Canada), based on raw NMR spectra. The regions of the raw NMR spectra that were used to build each chemometric model were selected using one of the built-in optimization routines within the OPUS™ software.

### Example 1: T₁-Weighted T₂ Low-Field NMR Signals for Different Hydrocarbon Streams

Example 1 qualitatively demonstrated how different hydrocarbon streams exhibited faster NMR signal relaxation rates with increasing boiling point temperature (i.e. naphtha, light gas oil, heavy gas oil, bitumen, followed by >524° C. material). The relationship between relaxation behavior and boiling temperature supported a rational chemometric approach based on NMR signals to predict boiling point information of extra heavy oil samples. Since MCR content generally increases with boiling point temperature of liquid hydrocarbon streams as known in the art, this also supported a rational chemometric approach based on NMR signals to predict MCR content of extra heavy oil samples.

T₁-weighted T₂ low-field NMR measurements were made on test samples of diluent naphtha, hydrotreated light gas oil, hydrotreated heavy gas oil, Unitar bitumen, >524° C. hydrocarbon material isolated from bitumen, and solid fluid coke particles produced in a fluid coker. The different compositions were stored in polytetrafluroethylene (PTFE) vials during testing. The NMR measurement parameters were such that there were 100 transverse relaxation echoes spaced 0.3 ms apart, acquired at 28 T₁ points, exponentially spread from 0.1 ms through 3000 ms, and the final stretch of T₂ measurement comprises 500 echoes spaced 0.2 ms apart, with 4 scans averaged together to improve the signal to noise ratio, using a probe with a dead-time of 0.0277 ms, resulting in a measurement time of less than 3 minutes.

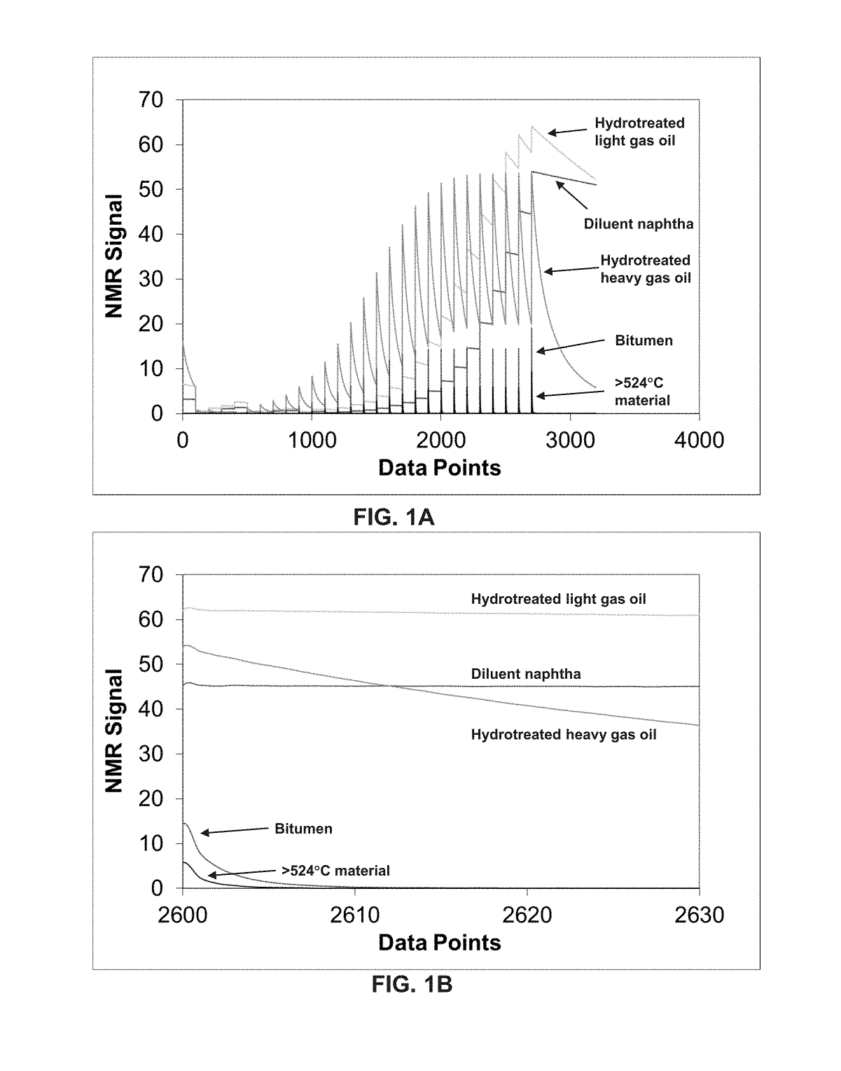

FIGS. 1A and 1B show T₁-weighted T₂ low-field NMR signals for the different hydrocarbon streams. The NMR signal intensity for the relatively viscous, higher boiling point temperature bitumen and the >524° C. hydrocarbon material was quite small in comparison with the NMR signal intensity for the relatively less viscous, lower boiling point naphtha, light gas oil, and heavy gas oil. It is believed that this is because a significant portion of the bitumen and >524° C. hydrocarbon material relaxed relatively quickly (e.g., in microseconds), and therefore had already relaxed by the time the measurement began. Not shown in FIGS. 1A and 1B is that solid fluid coke particles produced virtually no signal. It is believed that this is because the hydrogen nuclei within the solid fluid coke particles are highly immobile, and thus relaxed before the NMR instrument began measuring any signal. This supported a rational approach that measures the solids content by difference from the measured NMR signal produced by liquid hydrocarbon components of the sample that are measureable.

### Example 2: FID Relaxation NMR Signals for Different Mixtures Comprising Bitumen, >524° C. Hydrocarbon Material, and Solid Fluid Coke Particles

Example 2 demonstrated that mixtures of different compositions commonly encountered in extra heavy oil streams during upgrading of bitumen exhibited different NMR signals. This further supported a rational chemometric approach based on NMR signals to predict the solids content, boiling point information, and MCR content of extra heavy oil samples.

Free induction decays (FID) NMR measurements were made on different mixtures: Unitar bitumen; a 1:1 by weight mixture of Unitar bitumen and >524° C. hydrocarbon material; a 1:1 by weight mixture of Unitar bitumen and solid fluid coke particles; >524° C. hydrocarbon material; 1:1 by weight mixture of >524° C. hydrocarbon material and solid fluid coke particles. The test parameters were as follows: acquisition window after pulse: 0.1 ms; and data points: 150.

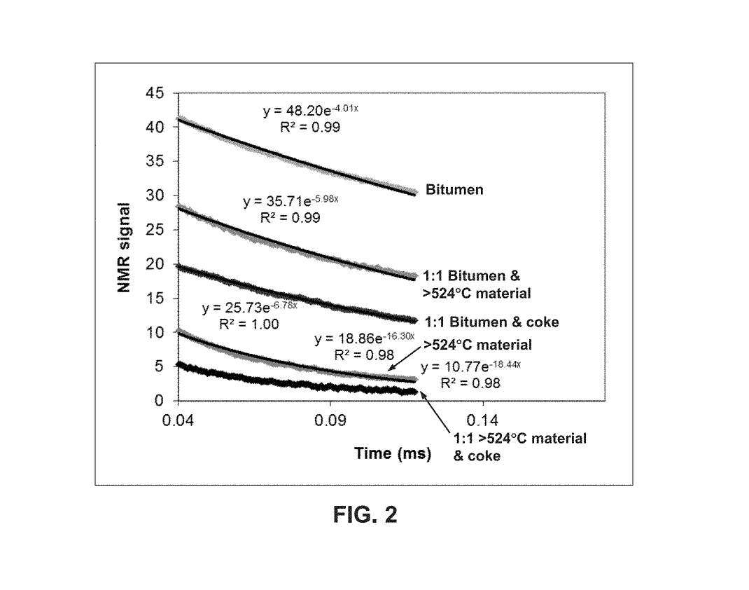

FIG. 2 shows that the different mixtures exhibited significantly different FID-relaxation NMR signals. Unitar bitumen relaxed more slowly than >524° C. hydrocarbon material, and a 1:1 mixture of the two relaxes at an intermediate rate. Unitar bitumen provides more NMR signal intensity than >524° C. hydrocarbon material. It is believed that this is partly because less of the Unitar bitumen has relaxed before the measurement of the NMR signal began. Further, replacing half of either Unitar bitumen or >524° C. hydrocarbon material with an equal weight of solid fluid coke particles drops the signal intensity by about half, while also somewhat increasing the relaxation rates due to restricted diffusion and/or surface relaxation effects as known in the art.

### Example 3: T₁-Weighted T₂ Low-Field NMR Signals for Simulated Coker Scrubber Slurry Samples

Example 3 demonstrated the ability to develop to chemometric PLS calibration models to predict boiling point information and solid fluid coke content for simulated coker scrubber slurry samples of known composition.

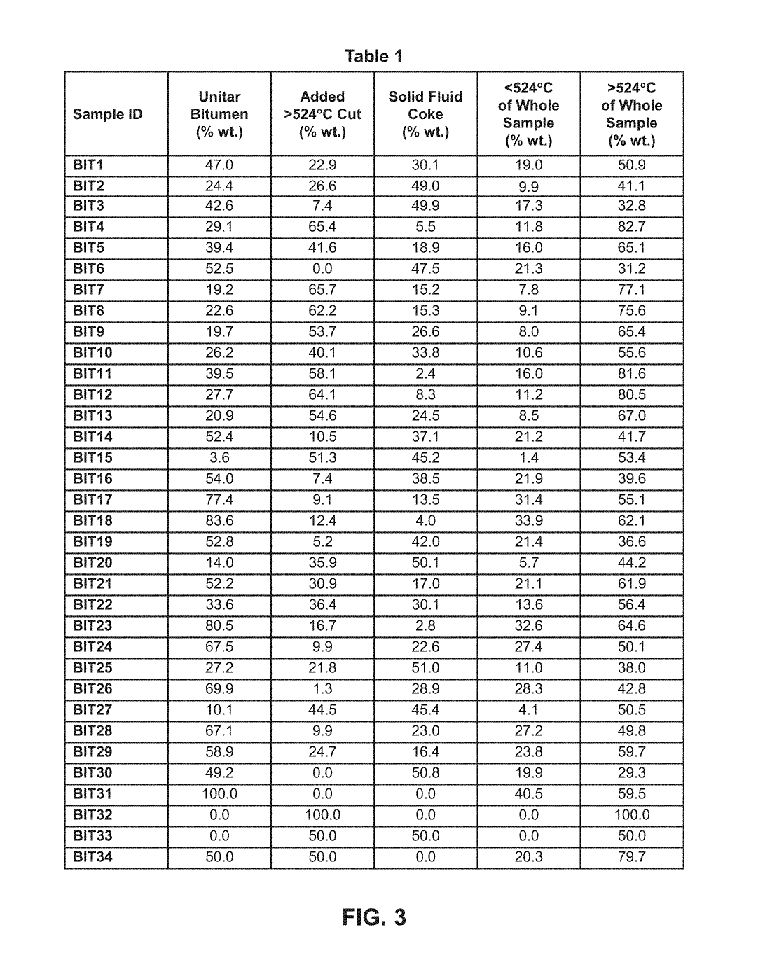

In FIG. 3, Table 1 summarizes the composition of thirty-four (34) simulated coker scrubber slurry samples. The sample weights were 4.02±0.24 grams. The samples were prepared using varying amounts of Unitar bitumen, >524° C. hydrocarbon material, and solid fluid coke particles, that were heated and stirred together in polytetrafluroethylene (PTFE) vials before they had cooled. The amounts of the components in samples BIT1 to BIT29 were somewhat randomly varied, while at least one component was purposely omitted from samples BIT30 to BIT34. In Table 1, the amount of <524° C. hydrocarbon content and >524° C. hydrocarbon content in each whole sample was calculated based on the known percentage (˜59.5%) of >524° C. hydrocarbon material in the Unitar bitumen and the known amount of added >524° C. hydrocarbon material. The samples were stored in the PTFE vials during testing.

T₁-weighted T₂ low-field NMR measurements were made on the samples. The ‘samples were heated to 40° C. at the time of testing, which was the same as the NMR system's probe temperature. The NMR measurement parameters were such that there were 200 transverse relaxation echoes spaced 0.04 ms apart, acquired at 28 T₁ points, exponentially spread from 5 ms through 200 ms, with 16 scans averaged together to improve the signal to noise ratio, using a probe with a dead-time of 0.0097 ms, resulting in a measurement time of less than 1 minute. In comparison with Example 1, the shorter dead-time of the NMR probe in this Example 3, was used to observe as much of the fast relaxing signal as possible.

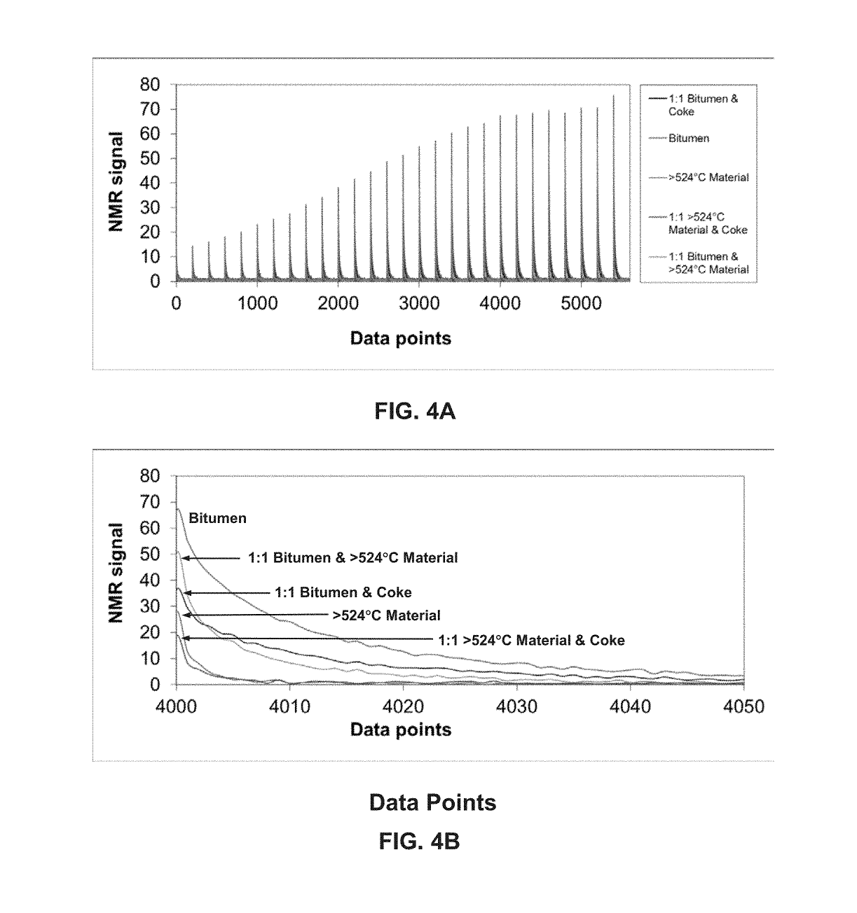

FIGS. 4A and 4B show the raw T₁-weighted T₂ low-field NMR signals, plotted by data point, for samples BIT30 to BIT34 in Table 1 of FIG. 3. FIGS. 4A and 4B illustrate that relaxation rates increase with increasing >524° C. content. Further, the NMR signal intensity decreases with increasing solid fluid coke particle content.

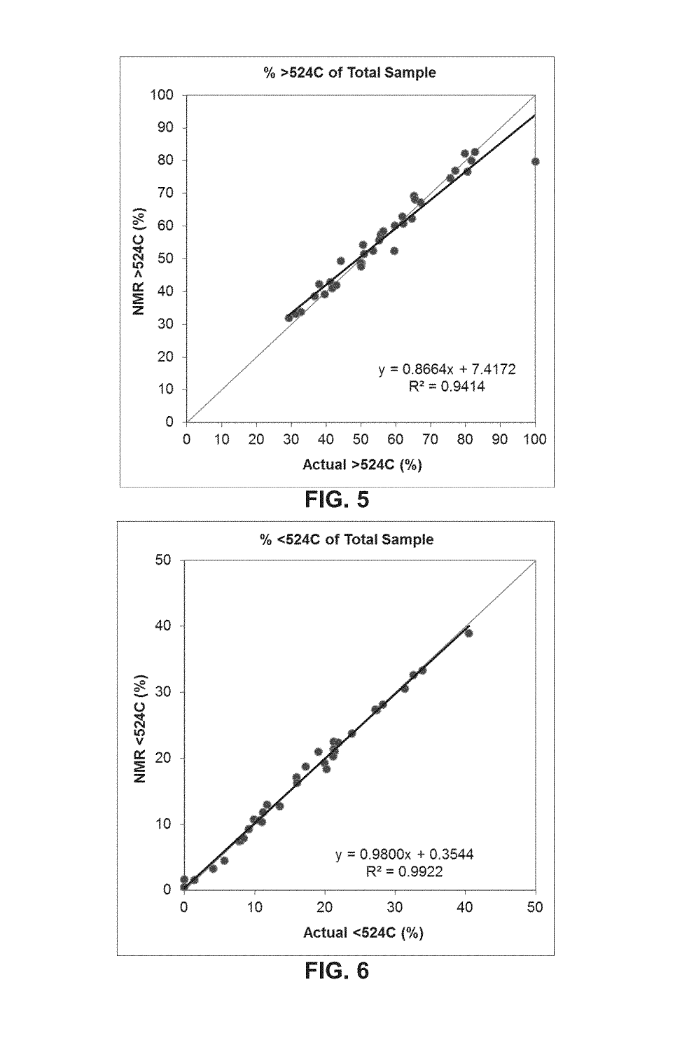

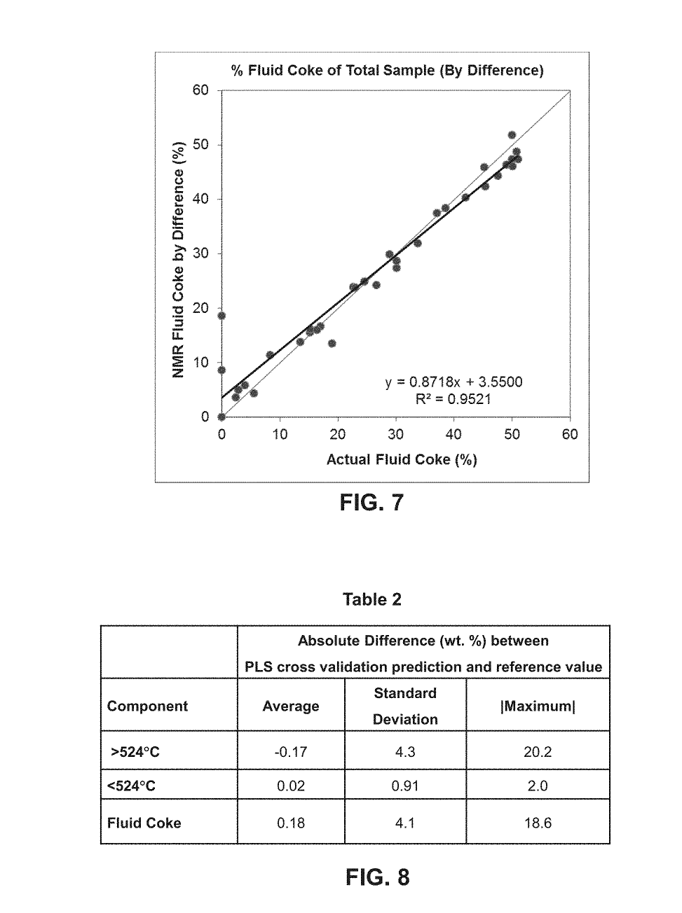

FIGS. 5, 6, and 7 compare the >524° C. hydrocarbon content, <524° C. hydrocarbon content, solid fluid coke particle content, respectively, predicted by PLS cross validation models based on the T₁-weighted T₂ low-field NMR signals for the samples of Table 1 in FIG. 3, with the known amounts in the prepared samples. In FIG. 8, Table 2 summarizes the statistical measures of the difference between the PLS cross validation predicted results and known amounts of these components. Very good agreement was observed between the predicted and reference amounts, with only a couple of outliers (e.g., BIT32 of 100% wt.>524° C. hydrocarbon material and, to a lesser degree, sample BIT33 of 50% wt.>524° C. hydrocarbon material and 50% wt. solid fluid coke particles). These outliers relaxed more quickly than the other samples and thus were poorly represented by the remaining samples in the cross validation models.

### Example 4: T₁-Weighted T₂ Low-Field NMR Signals for Actual Coker Scrubber Slurry Samples

Example 4 demonstrated the ability to develop chemometric PLS calibration models to predict the solids content, boiling point information, and MCR content of actual coker scrubber slurry test samples, with appreciably good accuracy to conventional analytical methods.

Sixty-four (64) coker scrubber slurry samples were collected from three fluid cokers operated as part of Syncrude Canada Ltd.'s oil sands upgrading plant near Fort McMurray, Canada. To generate samples with a wider variety of compositions, some of the samples were blended with each other to generate an additional 60 blended samples, for a total of 124 samples.

Reference properties for each sample were determined using analytical methods as follows. The solids content was determined on sub-samples of the sample by extraneous matter vacuum filtration following dilution with toluene, and reported on a whole sample basis. Sub-samples of these samples were hot filtered through 150 mm diameter Whatman™ GF/A filters (˜1.6 micron pore size) in an oven at 100-140° C. until sufficient filtrate material for further analysis was obtained. The resulting “liquid” filtrates were subjected to high temperature simulated distillation by gas chromatography in accordance with ASTM D7169 to determine the boiling point information of each sample. The resulting “liquid” filtrates were also subjected to micro carbon residue analysis in accordance with ASTM D-4530 to determine their MCR content.

Since the reference boiling point information and MCR content were determined using the “liquid” filtrate, they are initially determined on a “liquid only” basis. However, MCR content is typically reported on a “whole sample” basis. Accordingly, MCR content on a “whole sample” basis was also determined by adding the weight of solids determined by vacuum filtration to the weight of MCR determined on a “liquid only” basis, and dividing the sum by the whole sample weight.

The weight of non-solids, <524° C. hydrocarbon material, <524° C. hydrocarbon material, and non-MCR content could be calculated from the results of the above analytical methods. For example, consider a coker scrubber slurry test sample having a total weight of 10 g that was determined to have a solids content of 10% on a whole sample basis, a 15% MCR content on a liquid-only basis, and a 30%<524° C. hydrocarbon content and 70%>524° C. content on a liquid-only basis. The weight of the non-solids, non-MCR, <524° C. hydrocarbon content, and >524° C. hydrocarbon content in this test sample could be calculated as follows: non-solids=90% of 10 g=9 g; non-MCR=85% of 9 g=7.65 g; <524° C. material=30%×9 g=2.7 g; and >524° C. material=70% of 9 g=6.3 g.

Prior to measurement in the NMR system, the samples were heated to about 100-110° C. for at least 2 hours until they could be stirred vigorously, with emphasis on scraping material from the bottom of the container and pulling it to the top to re-suspend any settled solids. They were then sub-sampled into 16 mL glass vials with low-density polyethylene (LDPE) snap on lids (part number: 225536 from Wheaton Industries Inc., Millville, N.J., USA). The LDPE lids were removed prior to measurement in the NMR to avoid any hydrogen signal from the lid.

About ⅔ of the samples (referred to as “calibration samples”) were used to construct chemometric PLS calibration models based on NMR testing. The chemometric PLS calibration models were used to predict the properties of the remaining ⅓ of the test samples, which are referred to as “test set samples”.

T₁-weighted T₂ low-field NMR measurements were made on the calibration samples. All calibration samples were heated to the same 40° C. temperature as the NMR probe using a Techne Dri-Block DB-3D™ block heater. The calibration sample insertions into the NMR instrument were performed by a Duratech ASP-960™ autosampler (Noblesville, Ind., USA). The NMR measurement parameters were such that there were 200 transverse relaxation echoes spaced 0.04 ms apart, acquired at 28 T₁ points, exponentially spread from 5 ms through 200 ms, with 16 scans averaged together to improve the signal to noise ratio, using a probe with a dead-time of 0.0097 ms, resulting in a measurement time of less than 1 minute.

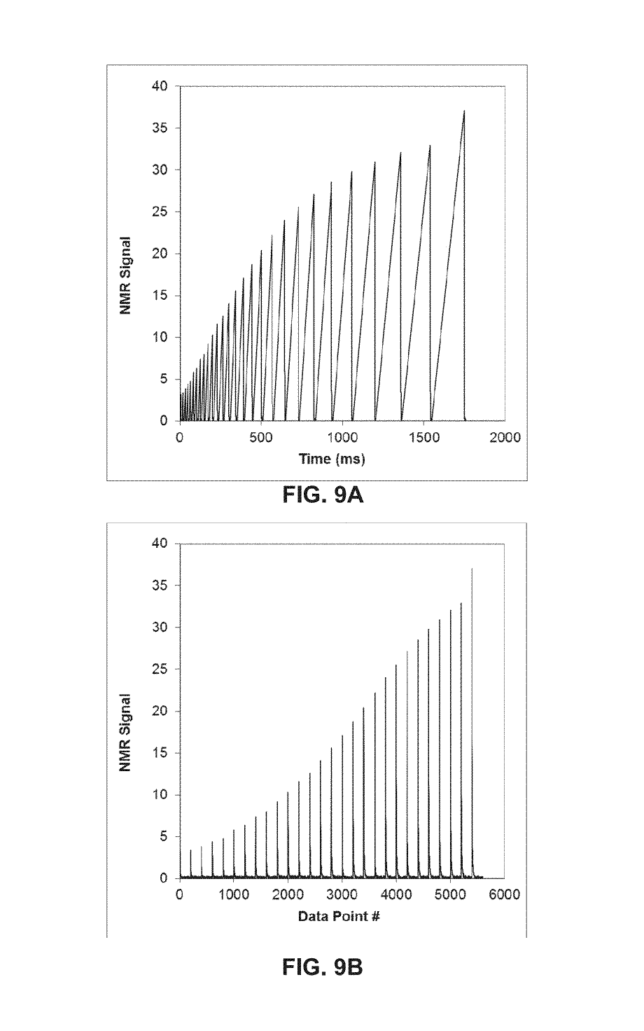

FIG. 9A shows the raw T₁-weighted T₂ low-field NMR signals, plotted by time, for fluid coker scrubber slurry calibration samples at 40° C.; FIG. 9B shows the T₁-weighted T₂ low-field NMR signals of FIG. 9A, plotted by data point. Further tests were performed at an autosampler heating block temperature of 70° C. to determine if higher sample temperatures produce better results, for example by reducing sample viscosity which reduces relaxation rates as known in the art, which would increase the amount of observable signal given the limitations of the NMR probe dead time.

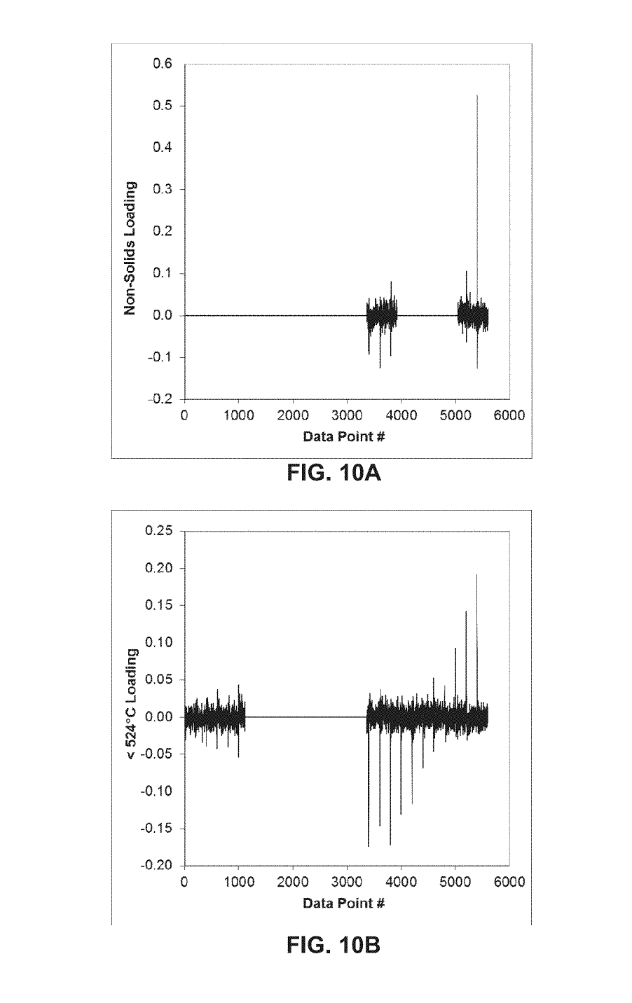

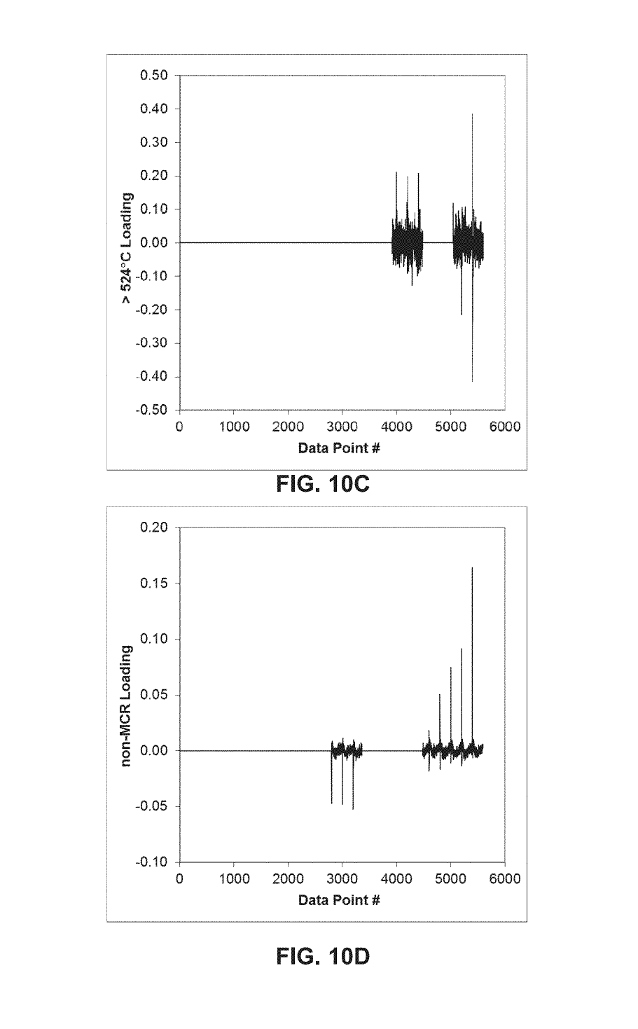

PLS chemometric calibration models were determined based on the NMR spectra and the reference values for the calibration samples. FIGS. 10A, 10B, 10C, and 10D show the PLS calibration model loading for determining the non-solids content, the <524° C. hydrocarbon content, the >524° C. hydrocarbon content, and the non-MCR content, respectively, from the NMR signals of FIG. 9B. In particular, the non-solids content reference values for the calibration samples were used to develop a non-solids PLS calibration model that could predict the solids content by difference, since the NMR produces minimal signal for solids. Similarly, non-MCR content reference values for the calibration samples were also used to build a PLS calibration model for the MCR content that could predict MCR content by difference. Chemometric PLS calibration models were also developed based directly on the weight of solids content and weight of MCR content, but were found to generally be less accurate. Efforts to develop calibrations for predicting solids content from FID signals rather than T₁-weighted T₂ signals were also unsuccessful.

In the following discussion, the comparison of properties predicted by the PLS calibration models with reference properties were based only on the test set sample results.

### Solids Content

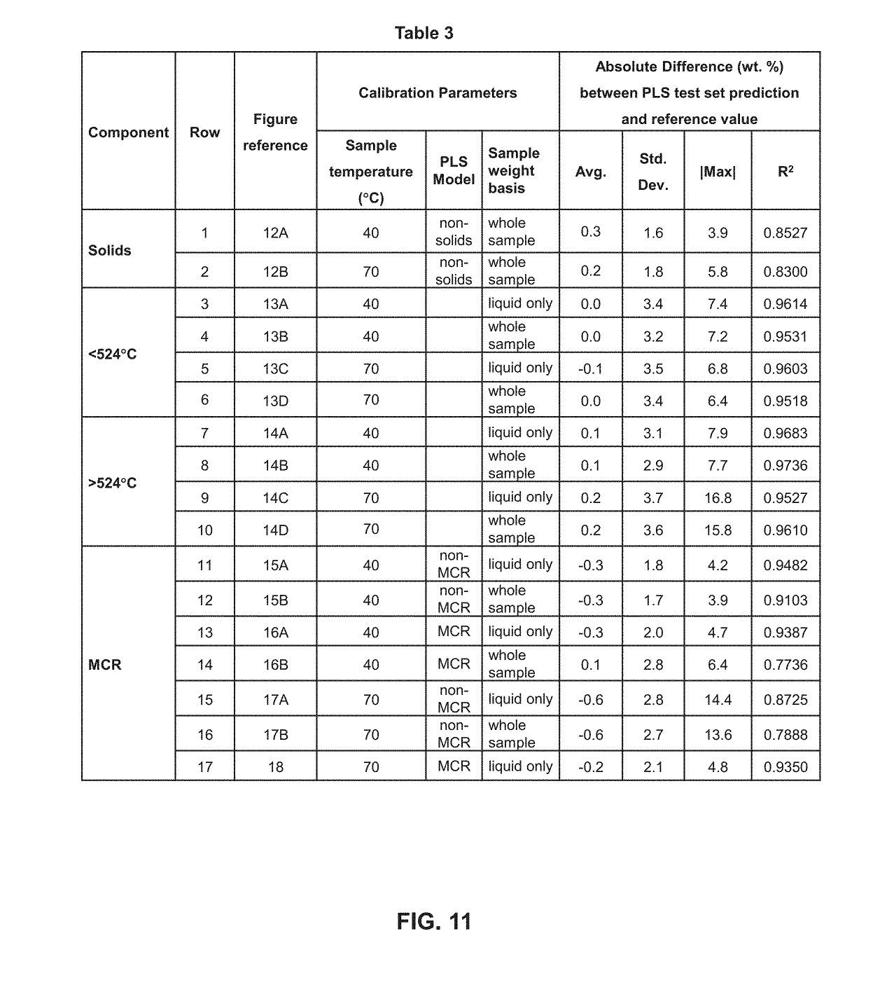

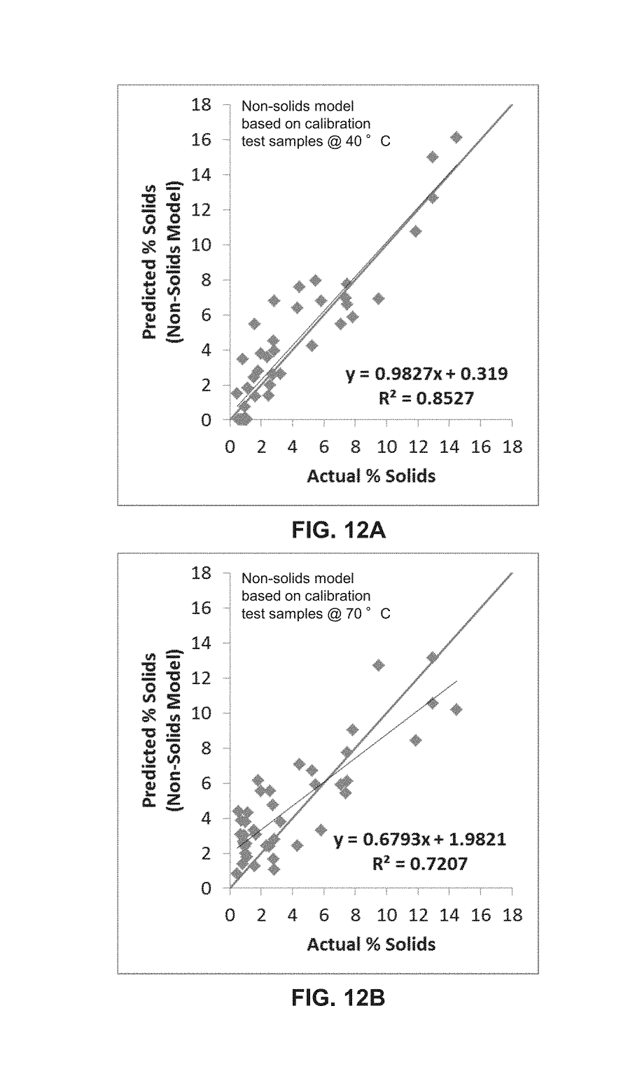

The solids content (on a whole sample basis) of the test samples was predicted using a non-solids PLS calibration model based on calibration samples at both 40° C. and 70° C., and compared with reference values determined by vacuum filtration analysis. In FIG. 11, Table 3, rows 1 and 2 summarize the statistical measures of the difference between the predicted and reference values, while FIGS. 12A and 12B compare the predicted and reference values. The results indicated that the PLS calibration models are able to provide a rough estimate of the solids content in the test samples. It is believed that the inaccuracy is attributable to the inability to measure portions of the signal from liquid hydrocarbons within the samples that relax so quickly that they cannot be measured during the dead time of the NMR probe.

### Boiling Point Information

The boiling point information of the test samples was predicted using PLS calibration models based on calibration samples at both 40° C. and 70° C., on both a whole sample and liquid only basis, and compared with reference values determined by high temperature simulated distillation on the “liquid” filtrate.

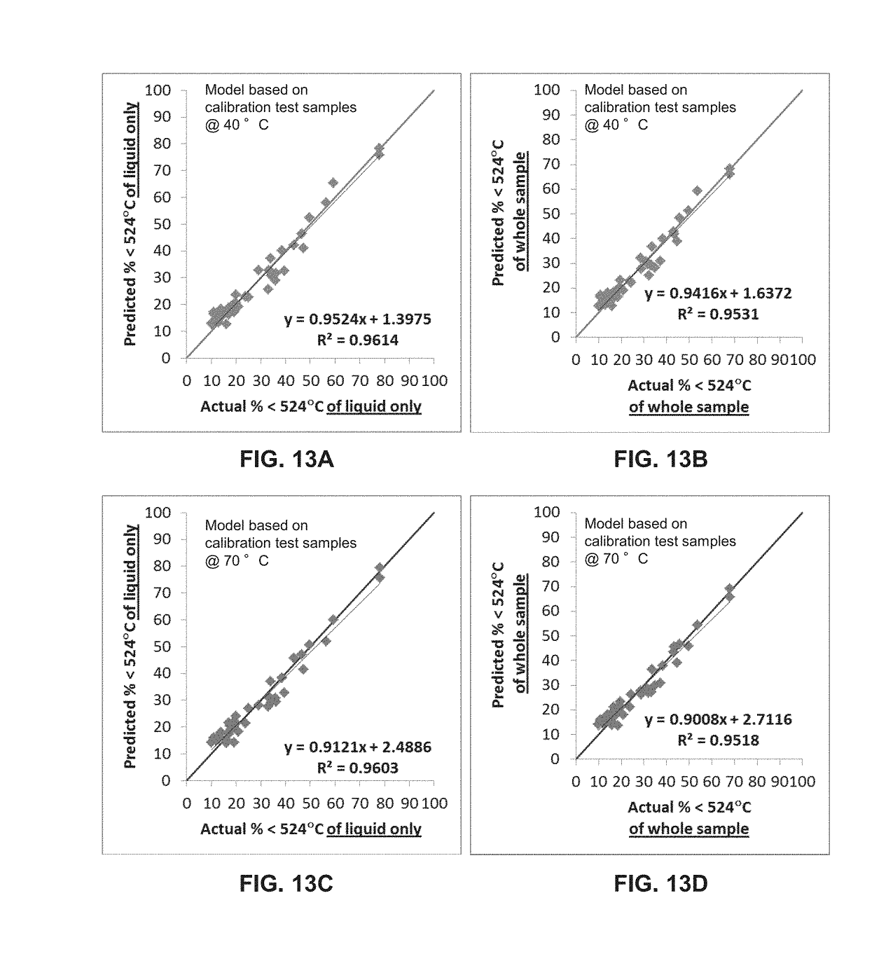

In FIG. 11, Table 3, rows 3 to 6 summarize the statistical measures of the difference between the predicted and reference values for <524° C. hydrocarbon content, while FIGS. 13A to 13D compare the predicted and reference values. The agreement between predicted and reference amounts of <524° C. hydrocarbon material was excellent irrespective of whether the temperature of the samples was 40° C. or 70° C.

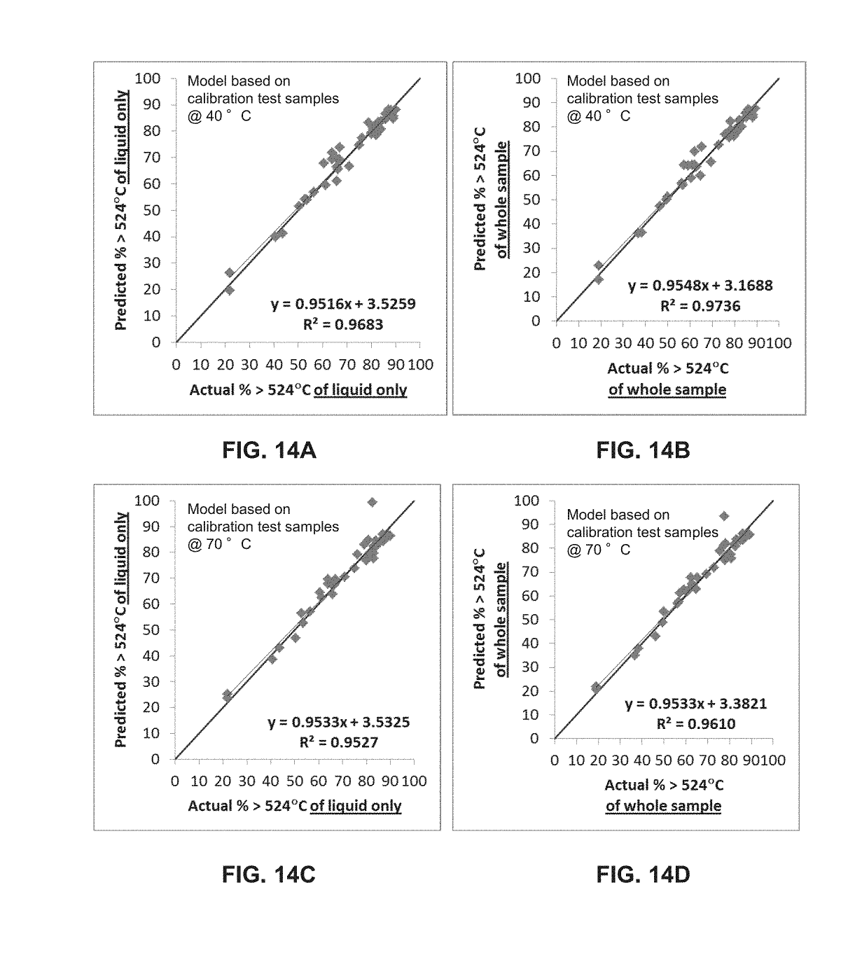

In FIG. 11, Table 3, rows 7 to 10 summarize the statistical measures of the difference between the predicted and reference values for >524° C. hydrocarbon content, while FIGS. 14A to 14D compare the predicted and reference values. The agreement between predicted and reference amounts of >524° C. hydrocarbon content was excellent irrespective of whether the temperature of the samples was 40° C. or 70° C.

### MCR Content

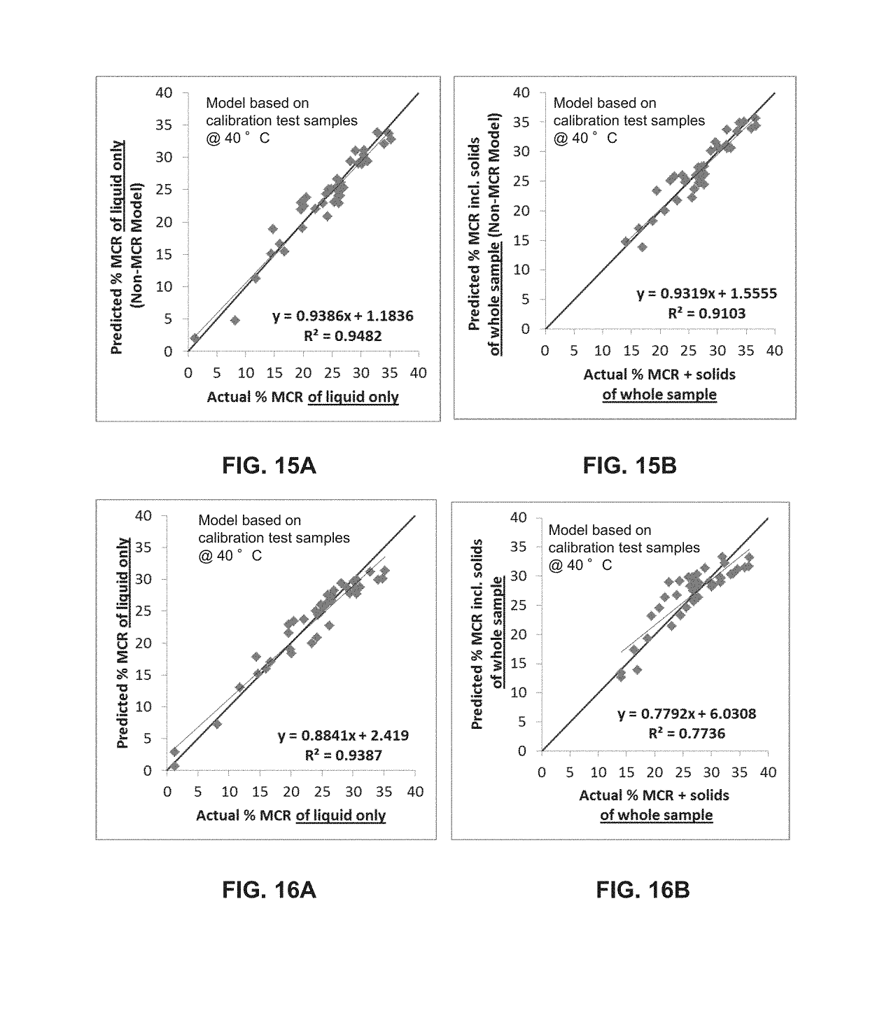

The MCR content was determined using PLS calibration models based on calibration samples at both 40° C. and 70° C., on both a whole sample and liquid only basis, and compared with reference values determined by micro carbon residue analysis of the “liquid” filtrate. In FIG. 11, Table 3, rows 11 to 17 summarize the statistical measures of the difference between the predicted and reference values of MCR content, while FIGS. 15A to 18 compare the predicted and reference values. Because the NMR was unable to provide a highly reliable prediction for the solids content in this Example 4, the amount of liquid fraction in each sample was calculated using the vacuum filtration results for the results reported on a “liquid only” basis. The Figures indicate that NMR provided good to excellent predictions of the MCR content for coker scrubber slurry samples or potentially other heavy oil samples of roughly similar composition. The non-MCR calibration models based on calibration samples at 40° C. provided better agreement than the other models.

### Repeatability

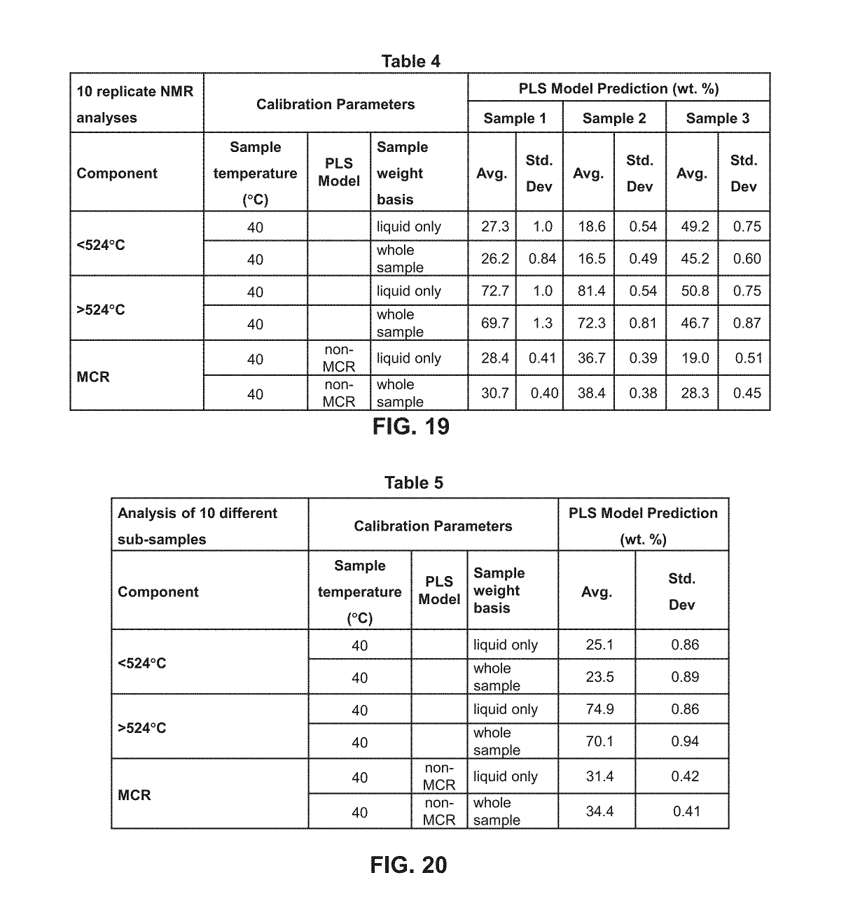

Three coker scrubber slurry samples at 40° C. were each analyzed 10 times using the NMR instrument to determine the repeatability of the predictions for <524° C. hydrocarbon content, >524° C. hydrocarbon content, and MCR content. In FIG. 19, Table 4 summarizes statistical measures of PLS chemometric calibration model predictions for the NMR analysis of each of the three samples. Overall, the NMR instrument repeatability is very good with absolute values of standard deviations of 1.3% or less. The use of the autosampler in the NMR instrument ensured that each sample is analyzed in a very consistent automated manner with minimal lab technician involvement.

In addition, a single fluid coker scrubber slurry sample was divided into 10 separate sub-samples at 40° C., each of which was analyzed using the NMR instrument to determine the repeatability of the measurement for <524° C. hydrocarbon content, >524° C. hydrocarbon content, and MCR content for different sub-samples. In FIG. 20, Table 5 summarizes statistical measures of PLS calibration model predictions for the NMR analysis of the 10 sub-samples. The sub-sampling repeatability is very good and comparable to the instrument repeatability in Table 4 (FIG. 19), suggesting that sample mixing and sub-sampling steps were not significantly contributing to the overall measurement error.

### Example 5: Effect of NMR Pulse Sequence and Sample Container

Example 5 demonstrated the ability to develop more accurate chemometric PLS calibration models to predict the solids content, boiling point information, and MCR content of coker scrubber slurry test samples of Example 4, by variations in NMR pulse sequence parameters and sample containers.

The test conditions and methodology were the same as those used in Example 4 (discussed above), except as follows. First, in an effort to achieve a more consistent sample temperature, an empty glass vial was placed above the sample vial in the glass NMR autosampler tube to help reduce heat loss through the top portion of the sample in the sample vial. Second, to improve the signal to noise ratio and more fully observe sample relaxation behavior, the NMR measurement parameters were adjusted such that there were 50 transverse relaxation echoes spaced 0.04 ms apart, acquired at 28 T₁ points, exponentially spread from 5 ms through 1000 ms, with 16 scans averaged together to improve the signal to noise ratio, using a probe with a dead-time of 0.0097 ms, resulting in a measurement time of less than 2 minutes. As compared with Example 4, significant improvements in the prediction of solids content and MCR content were observed.

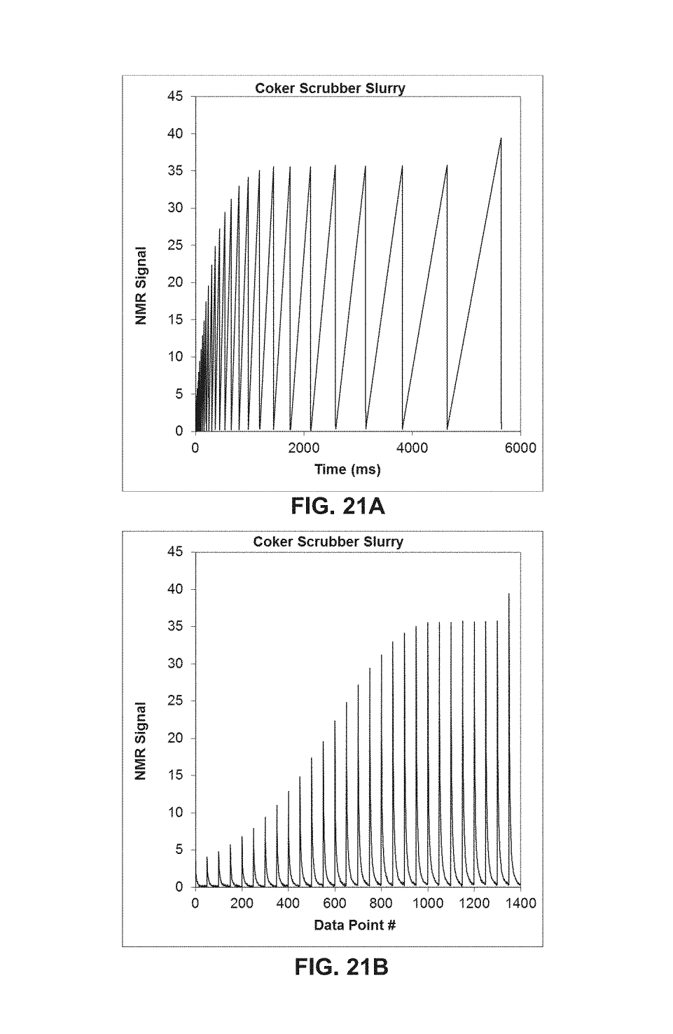

FIG. 21A shows the raw T₁-weighted T₂ low-field NMR signals, plotted by time; FIG. 21B shows the T₁-weighted T₂ low-field NMR signals of FIG. 21A, plotted by data point. Further tests were performed at an autosampler heating block temperature of 70° C. to determine if higher sample temperatures produce better results.

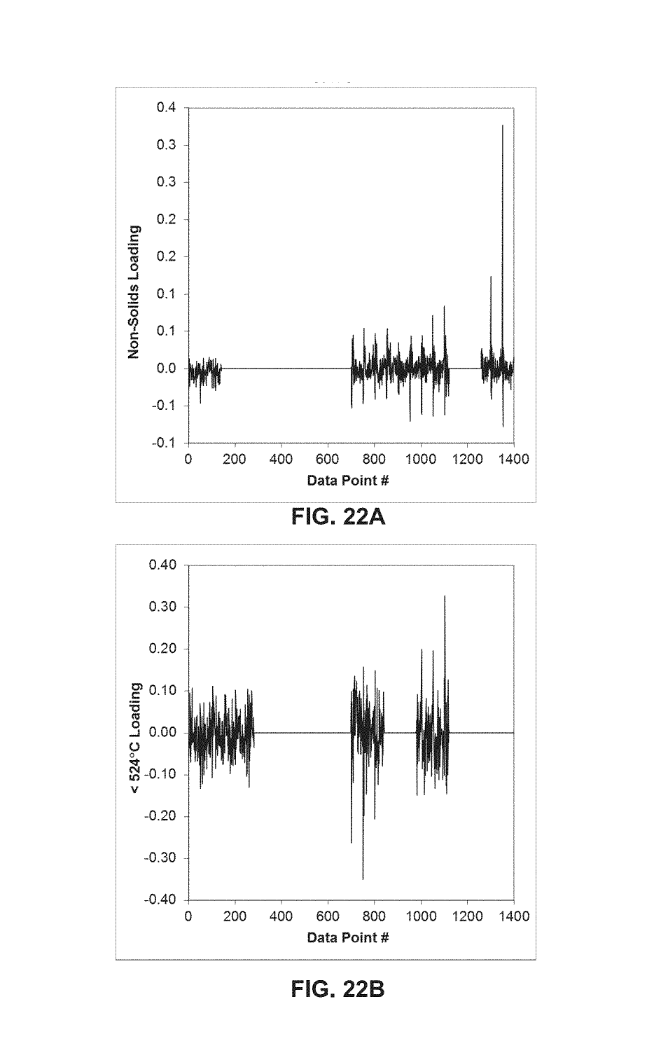

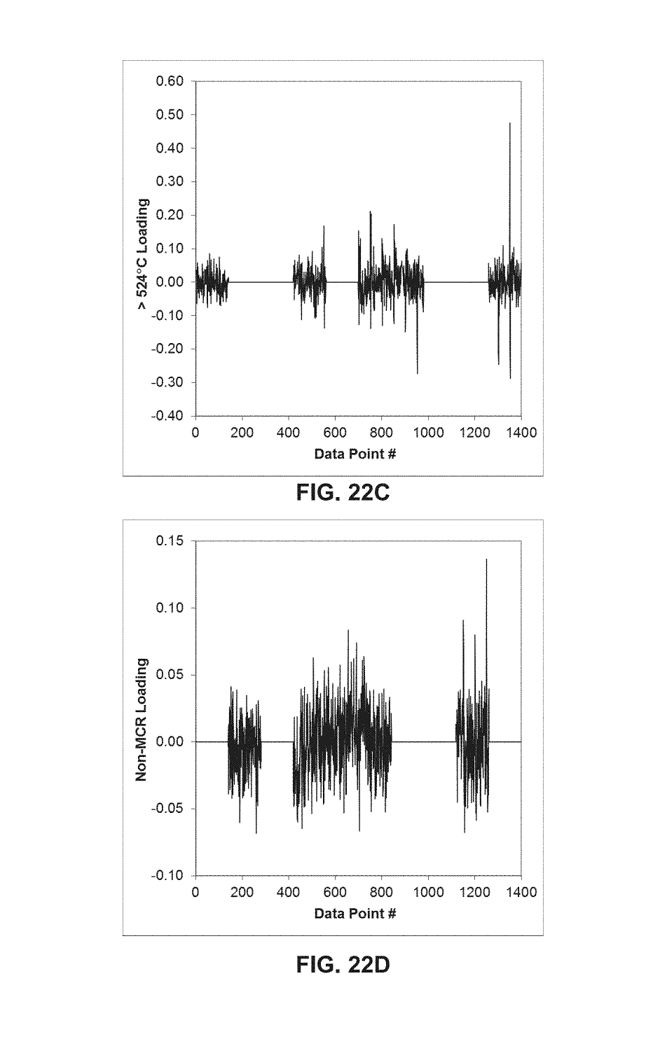

PLS chemometric calibration models were determined based on the NMR spectra and the reference values for the calibration samples. FIGS. 22A, 22B, 22C, and 22D show the PLS calibration model loading for determining the non-solids content, the <524° C. hydrocarbon material content, the >524° C. hydrocarbon material, and the non-MCR content, respectively, from the NMR signals of FIG. 21B.

In the following discussion, the comparison of properties predicted by the PLS calibration models with reference properties were based only on the test set samples.

### Solids Content

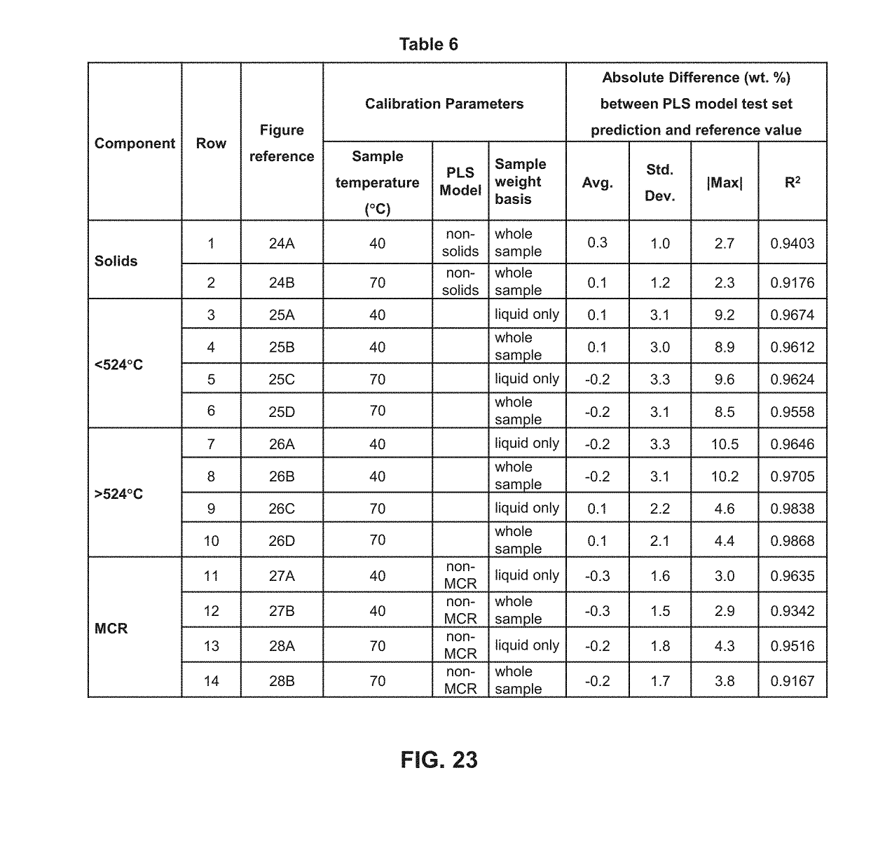

The solids content (on a whole sample basis) of the test samples was predicted using a non-solids PLS calibration model based on calibration samples at both 40° C. and 70° C., and compared with reference values determined by vacuum filtration analysis. In FIG. 23, Table 6, rows 1 and 2 summarize the statistical measures of the difference between the predicted and reference values, while FIGS. 24A and 24B compare the predicted and reference values. The results indicated that the PLS calibration models are able to provide a reasonable estimate of the solids content in the test set samples, with minimal outliers at % solids concentrations higher than ˜5%. This is an improvement over the results in Example 4 (see In FIG. 11, Table 3, rows 1 to 2, and FIGS. 12A and 12B). Further improvements in the accuracy of the % solids prediction may be possible with better temperature control at higher NMR analysis temperatures and/or by using an NMR probe with a shorter dead-time combined with logical adjustments of the pulse sequence parameters to record more of the fast relaxing NMR signal from the non-solid components within the samples.

### Boiling Point Information

The boiling point information of the test samples was predicted using PLS calibration models based on calibration samples at both 40° C. and 70° C., on both a whole sample and liquid only basis, and compared with reference values determined by high temperature simulated distillation on the “liquid” filtrate.

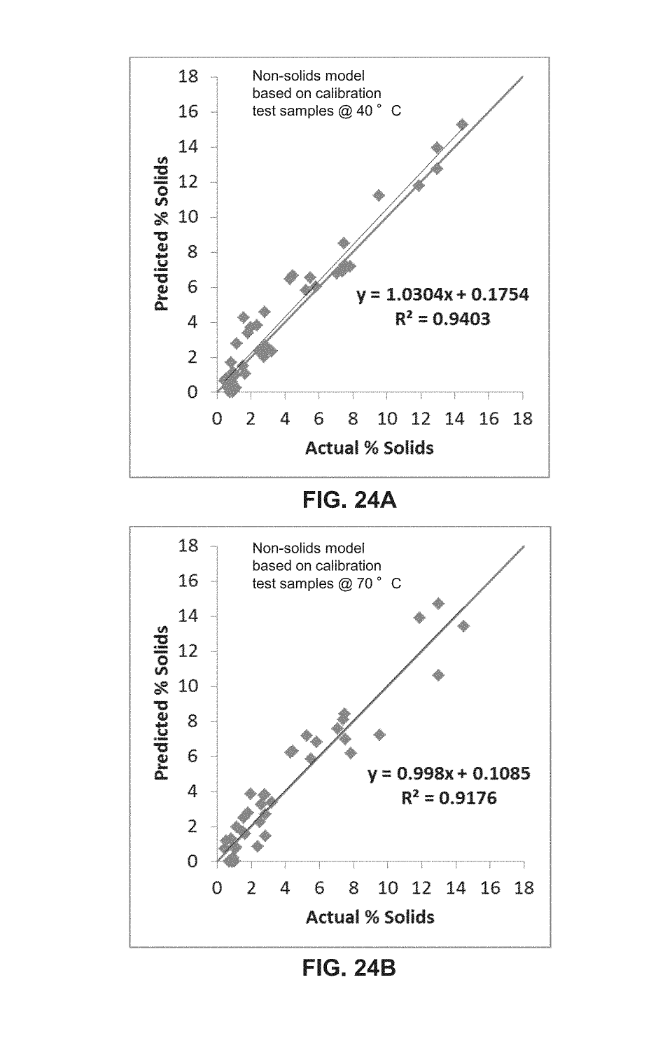

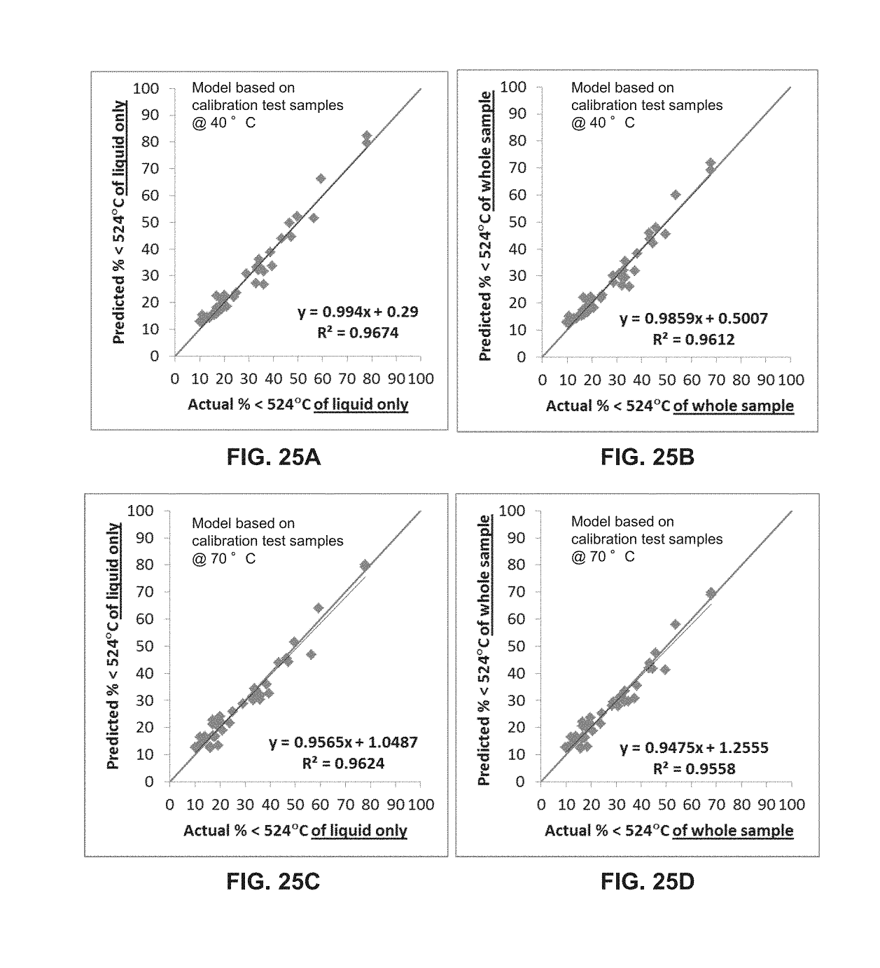

In FIG. 23, Table 6, rows 3 to 6 summarize the statistical measures of the difference between the predicted and reference values for <524° C. hydrocarbon content, while FIGS. 25A to 25D compare the predicted and reference values. The results are either comparable or better than the results in Example 4 (see FIG. 11, Table 3, rows 3 to 6, and FIGS. 13A to 13D).

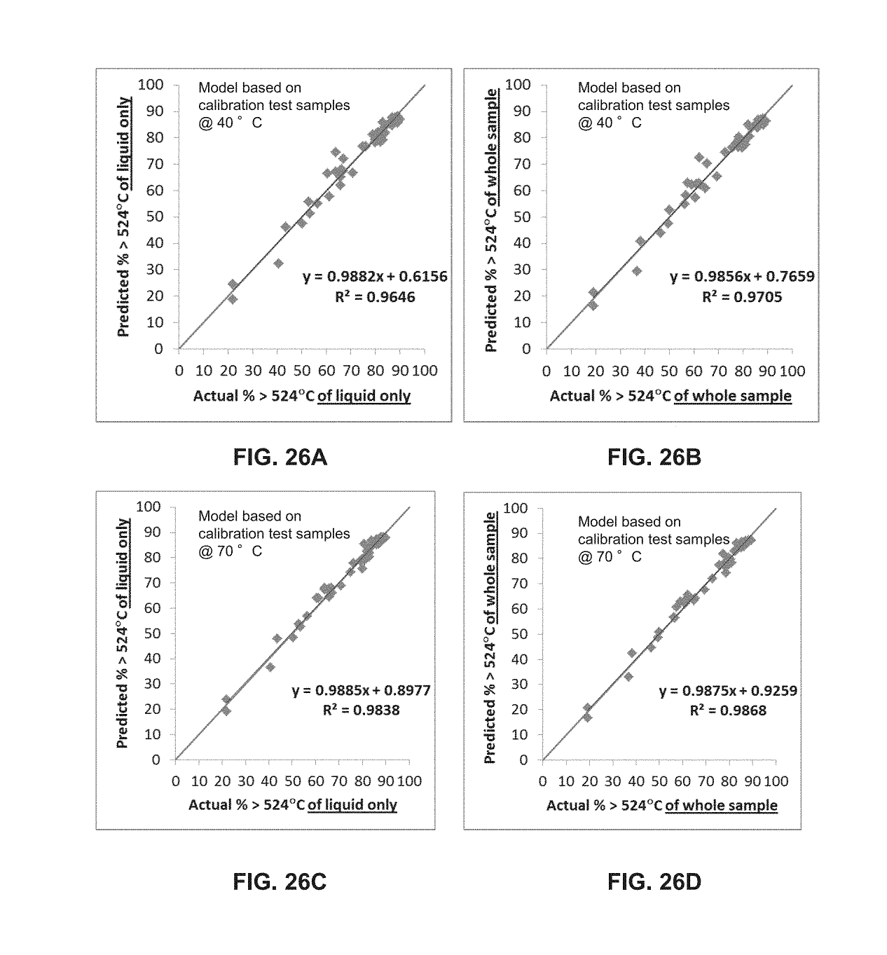

In FIG. 23, Table 6, rows 7 to 10 summarize the statistical measures of the difference between the predicted and reference values for >524° C. hydrocarbon content, while FIGS. 26A to 26D compare the predicted and reference values. The results are either comparable or better than the results in Example 4 (see FIG. 11, Table 3, rows 7 to 10, and FIGS. 14A to 14D).

### MCR Content

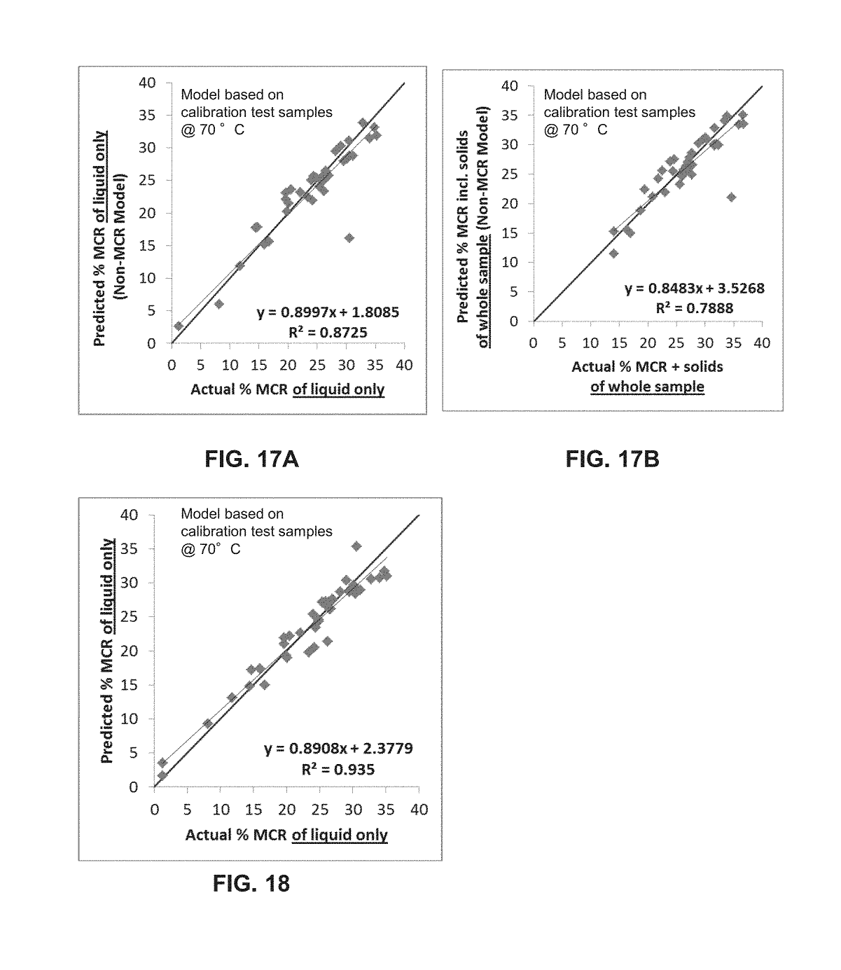

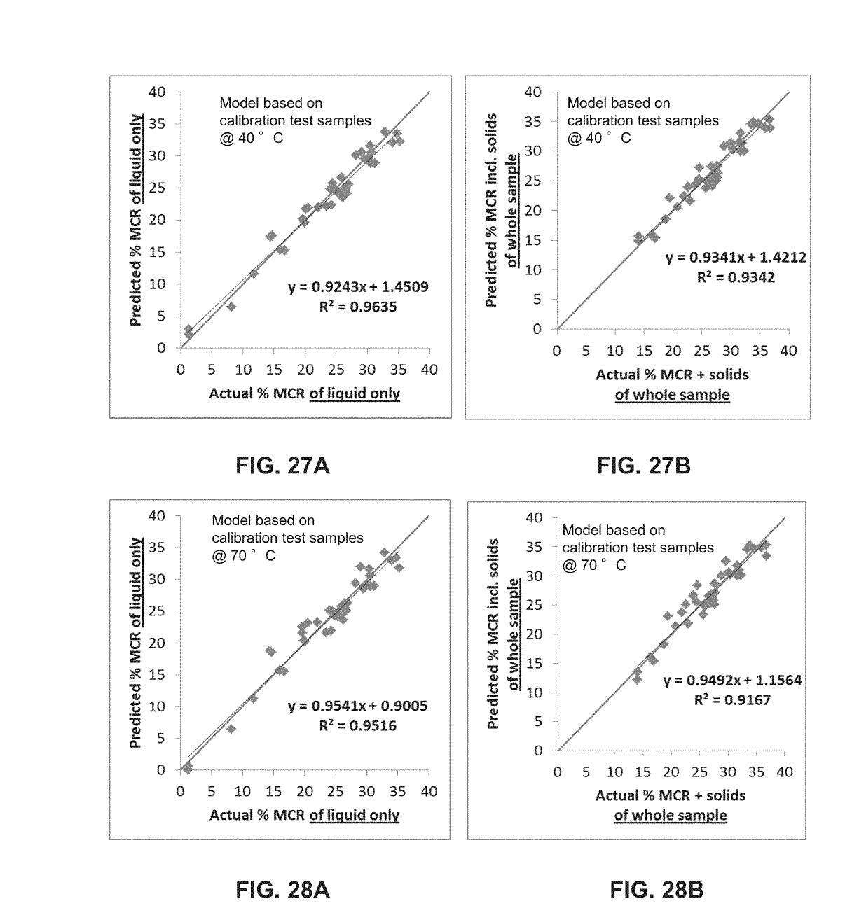

The MCR content was determined using PLS calibration models based on calibration samples at both 40° C. and 70° C., on both a whole sample and liquid only basis, and compared with reference values determined by carbon residue micro method analysis of the “liquid” filtrate. In FIG. 23, Table 6, rows 11 to 14 summarize the statistical measures of the difference between the predicted and reference values of MCR content, while FIGS. 27A to 28B compare the predicted and reference values. The Figures indicate that PLS calibration models provided good to excellent predictions of the MCR content for coker scrubber slurry samples or potentially other heavy oil samples of roughly similar composition. The results are an improvement over the results in Example 4 (see FIG. 11, Table 3, rows 11, 12, 15 and 16, and FIGS. 15A, 15B, 17A, 17B).

### Repeatability

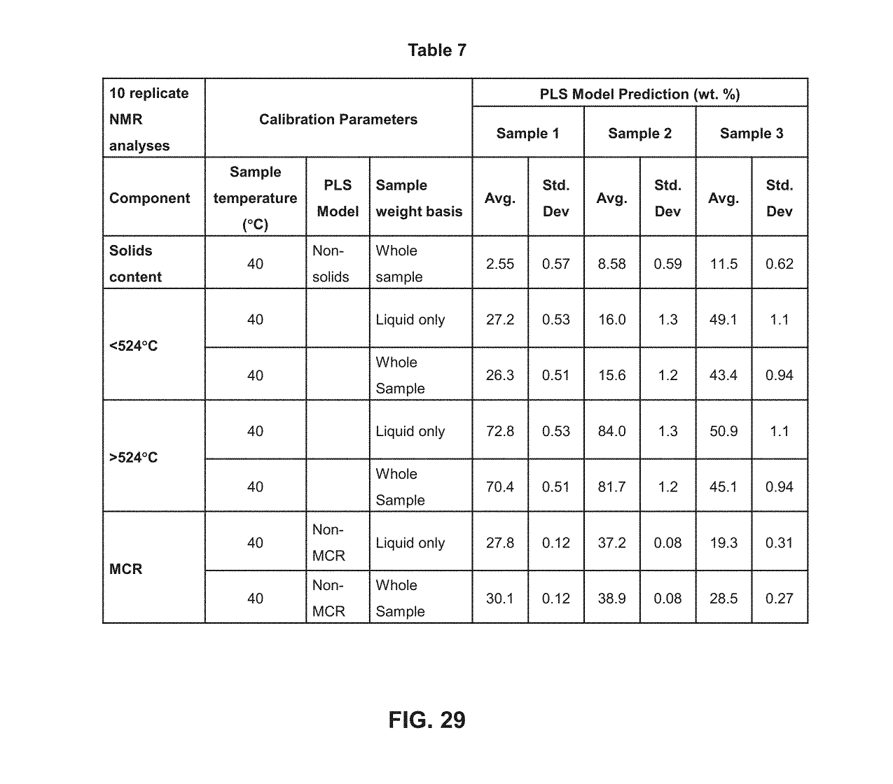

Three coker scrubber slurry samples at 40° C. were each analyzed 10 times using the NMR instrument to determine the repeatability of the prediction for the solids content, <524° C. hydrocarbon content, >524° C. hydrocarbon content, and MCR content. In FIG. 29, Table 7 summarizes statistical measures of PLS model predictions for the NMR analysis of each of the three samples. Overall, the NMR instrument repeatability is very good with absolute values of standard deviations of 1.3% or less, which is similar to the repeatability experienced for Example 4.

What is claimed:

1. A method for predicting at least one property of an extra heavy oil test sample using an NMR instrument, wherein the at least one property comprises one or a combination of a solids content, boiling point information, and a micro carbon residue (MCR) content, the method comprising the steps of:
(a) providing a plurality of extra heavy oil calibration samples, wherein each of the calibration samples has a reference value associated with each of the at least one property;
(b) subjecting each of the calibration samples to a T₁-weighted T₂ low-field NMR pulse sequence, and simultaneously recording T₁ and T₂ relaxation information to generate a NMR calibration data set;
(c) for each of the at least one property, determining a partial least squares (PLS) chemometric calibration model relating the NMR calibration data set to the reference values of the property of the calibration samples;
(d) subjecting the test sample to the T₁-weighted T₂ low-field NMR pulse sequence, and simultaneously recording T₁ and T₂ relaxation information to generate NMR test data; and
(e) predicting the at least one property of the test sample based on the PLS calibration model and the NMR test data.

2. The method of claim 1, wherein the extra heavy oil test sample is obtained from a process stream for upgrading bitumen recovered from oil sands ore to lighter crude oil.

3. The method of claim 2, wherein the extra heavy oil test sample comprises a fluid coker scrubber slurry.

4. The method of claim 2, wherein the extra heavy oil test sample comprises a residue of an atmospheric distillation column, a residue of a vacuum distillation column, or a residue of an ebullated bed hydrocracking reactor.

5. The method of claim 1, wherein the method further comprises the steps of selecting the associated reference values for the at least one property, and preparing the calibration samples with the selected associated reference values.

6. The method of claim 1, wherein the at least one property comprises the solids content.

7. The method of claim 6, wherein determining the PLS calibration model comprises relating reference values for non-solid content in the calibration samples to the NMR T₁-weighted T₂ signal data indicative of non-solids content in the calibration samples, and wherein predicting the solids content of the test sample comprises subtracting the non-solids content in the test sample as predicted by the PLS calibration model from a weight of a whole of the test sample.

8. The method of claim 6, whether the method further comprises the step of determining the reference values for the solids content of the calibration samples by subjecting the calibration samples or a subsample thereof to extraneous matter vacuum filtration following dilution with an appropriate solvent to reduce the viscosity of the calibration samples or subsamples thereof.

9. The method of claim 1, wherein the at least one property comprises boiling point information.

10. The method of claim 9, wherein the boiling point information comprises a concentration, by weight, of hydrocarbon content having a boiling point temperature less than or greater than at least one specified temperature.

11. The method of claim 10, wherein the at least one specified temperature comprises 524° C.

12. The method of claim 9, whether the method further comprises the step of determining the reference values for the boiling point information of the calibration samples by subjecting the calibration samples or subsamples thereof to simulated distillation analysis by gas chromatography.

13. The method of claim 1, wherein the at least one property comprises the MCR content.

14. The method of claim 13, wherein determining the PLS calibration model comprises relating reference values for non-MCR content in the calibration samples to the NMR T₁-weighted T₂ signal data indicative of non-MCR content in the calibration samples, and wherein predicting the MCR content of the test sample comprises subtracting the non-MCR content as predicted by the PLS calibration model from a weight of a whole of the test sample.

15. The method of claim 13, wherein the method further comprises the step of determining the reference values for the MCR content of the calibration samples by subjecting the calibration samples or subsamples thereof to micro carbon residue analysis by measuring a residual weight of the calibration samples or subsamples thereof after evaporation and pyrolysis of hydrocarbon material at 500° C. under a nitrogen atmosphere.

16. The method of claim 1, wherein the at least one property comprises the combination of solids content, boiling point information, and MCR content.

17. The method of claim 1, wherein:
(a) the method further comprises removing at least a fraction of solids from the calibration samples to produce liquid portions of the calibration samples;
(b) step (b) of claim 1 comprises subjecting the liquid portions of the calibration samples to the T₁-weighted T₂ low-field NMR pulse sequence;
(c) the method further comprises removing at least a fraction of solids from the test sample to produce a liquid portion of the test sample;
(d) step (d) of claim 1 comprises subjecting the liquid portion of the test sample to the T₁-weighted T₂ low-field NMR pulse sequence; and
(e) step (e) of claim 1 comprises predicting the at least one property of the test sample for the liquid portion of the test sample.

18. The method of claim 17, wherein the at least one property comprises the boiling point information or the MCR content.

19. The method of claim 17, wherein the liquid portions of the calibration samples comprise liquid filtrates produced by hot filtration of the calibration samples, and wherein the liquid portion of the test sample comprises the liquid filtrate produced by hot filtration of the test sample.

20. The method of claim 1, wherein when the calibration samples and the test sample are subjected to the T₁-weighted T₂ low-field NMR pulse sequence, the calibration samples and the test samples are contained in glass containers.

21. The method of claim 20, wherein the glass container comprises a lower part containing the calibration sample or the test sample, and an upper part placed over a top of the lower part to prevent heat loss from a top portion of the calibration sample or the test sample.

22. The method of claim 1, wherein the calibration samples, the test sample, and an NMR probe of the NMR instrument are heated to a temperature of at least 40° C., when the calibration samples and the test sample are subjected to the T₁-weighted T₂ low-field NMR pulse sequence.

23. The method of claim 22, wherein the temperature is at least 70° C.

24. The method of claim 1, wherein the T₁-weighted T₂ low-field NMR pulse sequence is such that there are 50 transverse relaxation echoes spaced 0.04 ms apart, acquired at 28 T₁ points, exponentially spread from 5 ms through 1000 ms, with 16 scans averaged together to improve the signal to noise ratio, resulting in a measurement time of less than 2 minutes.
